(19) United States
(12) Patent Application Publication
(10) Pub. No.: US 2009/0035807 A1
(43) Pub. Date: Feb. 5, 2009

(54) ANALYSIS OF POLYPEPTIDE PRODUCTION

(75) Inventors: Joseph Edward McCellan, Weston, MA (US); Jason Craig Rouse, Londonderry, NH (US); Jason Eric Lieske, Kinston, NH (US); Javad M. Zadei, Bedford, NH (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/182,473

(22) Filed: Jul. 30, 2008

Related U.S. Application Data
(60) Provisional application No. 60/953,176, filed on Jul. 31, 2007.

(51) Int. Cl. C12Q 1/02

(52) U.S. Cl. 435/29

(57) ABSTRACT

The present invention is directed to methods of evaluating a process to produce a polypeptide preparation or evaluating an element of a process to produce a polypeptide preparation. The methods comprise providing a sample comprising the polypeptide, wherein the sample has not been subjected to affinity chromatography, and subjecting the sample to liquid chromatography. The sample may be a process bioreactor sample. The methods of the invention may be used to determine a value of a characteristic of the sample. The methods of the invention provide a real time or near real-time information regarding the process to produce a polypeptide.

### CROSS REFERENCE TO RELATED APPLICATIONS

This application claims priority to U.S. Provisional Application No. 60/953,176, filed Jul. 31, 2007, the disclosure of which is incorporated by reference herein in its entirety.

### FIELD OF THE INVENTION

The invention relates to methods of analyzing cell culture and process bioreactor samples containing a polypeptide of interest, and particularly the use of liquid chromatography such as high performance liquid chromatography (HPLC) to control or monitor the production of a polypeptide preparation.

### BACKGROUND OF THE INVENTION

Therapeutic polypeptide preparations produced using recombinant technologies are generally complex heterogeneous mixtures. The heterogeneity of these preparations may derive from variation in the amino acid sequence, stoichiometry, and a variety of post translational modifications such as proteolytic cleavage, oxidation and/or deamination and variable glycosylation. For example, the sugar structures attached to an amino acid backbone of a polypeptide can vary structurally in many ways including, sequence, branching, and sugar content. The heterogeneity of polypeptide preparations can potentially affect the product definition, consistency, potency and purity of the preparation.

Production parameters used at various stages of polypeptide production including expression, purification and storage can greatly affect the properties of the polypeptide preparation that is produced. To date, the creation of recombinantly produced polypeptide drugs having defined characteristics, whether an attempt to produce a generic version of an existing polypeptide drug or to produce a second generation or other polypeptide preparation having improved or desirable characteristics, has been scientifically challenging. This is due, in part, to the lack of methods that allow real-time or near real-time analysis during the production of polypeptide preparations.

### SUMMARY OF THE INVENTION

Methods described herein provide analytical techniques for evaluating a process characteristic of cell culture samples and process bioreactor samples used in the production of polypeptide preparations, e.g., recombinant polypeptide preparations. For example, methods disclosed herein can be used to evaluate a process for the preparation of an antibody or antigen-binding fragment thereof. The use of liquid chromatography, e.g., high performance liquid chromatography (HPLC), allows for the analysis of cell culture and process bioreactor samples that have not been subjected to chromatography, e.g., affinity chromatography, prior to the analysis.

Accordingly, in one aspect, the disclosure features a method of evaluating a process to produce a polypeptide preparation, e.g., a recombinant polypeptide preparation, or evaluating an element of a process of producing a polypeptide preparation, e.g., a recombinant polypeptide preparation. The method includes providing a cell culture sample or process bioreactor sample comprising a polypeptide, wherein the cell culture sample or process bioreactor sample has not been subjected to chromatography, e.g., affinity chromatography, and subjecting the cell culture sample or process bioreactor sample to liquid chromatography (LC), e.g., reverse-phase liquid chromatography, size exclusion liquid chromatography, normal phase liquid chromatography, hydrophobic liquid chromatography, anion exchange liquid chromatography, to thereby determine the absence, presence, value or amount of a process characteristic in the sample. The method optionally includes providing a determination of whether a value determined for the process characteristic meets a preselected criterion, e.g., is present or is present in a preselected amount or range.

In a preferred embodiment, the liquid chromatography is capable of detecting a process characteristic present in the cell culture sample or process bioreactor sample at a concentration of about 0.007 μg/μL.

In some embodiments, the cell culture sample or process bioreactor sample has been conditioned prior to subjecting the sample to LC.

In a preferred embodiment, the method includes providing a comparison of the value determined for a process characteristic with preselected criterion, e.g., a reference value or values, to thereby evaluate the process for producing the polypeptide preparation, or an element of the process of producing the polypeptide preparation. In some embodiments, the method includes determining whether the test value is equal to or greater than the preselected criterion, if it is less than or equal to the preselected criterion, or if it falls within a range. In other embodiments, a test value, e.g., obtained by LC and/or mass spectrometry (MS), need not be a numerical value but may merely indicate whether the process characteristic or characteristics are present.

In some embodiments, the preselected criterion is the presence or amount of the process characteristic in a polypeptide preparation made by a different process or with a different element of the process than the process used to produce the polypeptide preparation being evaluated. In other embodiments, the preselected criterion is the presence or amount of the process characteristic present in a polypeptide preparation made by the same process as the polypeptide preparation being evaluated.

In one embodiment, when the preselected criterion is obtained by a different process, the process used to produce the cell culture sample or process bioreactor sample to be evaluated increases the amount of polypeptide preparation obtained as compared to the process used for the preselected criterion. In one embodiment, the process used to produce the cell culture sample or process bioreactor sample uses a different production parameter than the process used for the preselected criterion. Production parameters include: host cell type, selection of host subclones based on desired polypeptide preparation properties; regulation of host gene levels constitutive or inducible; introduction of novel genes or promoter elements; media components, e.g. media components described herein; physiochemical growth properties such as those described herein; growth vessel type (e.g. bioreactor type, T flask); cell density; and cell cycle. For example, the production parameter can be one or more of: a different host cell than the host cell used to produce the preselected criterion; the process used to produce the cell culture sample or process bioreactor sample has one or more media components which differ from the media component or components used to produce the preselected criterion; the cell or cell line used to produce the cell culture sample or process bioreactor sample has a different vector or vector elements than the cell or cell line used to produce the preselected criterion; a culture condition or culture conditions used to produce the cell culture sample or process bioreactor sample differ from a culture condition or conditions used to produce the preselected criterion.

In one embodiment, when the same procedure is used for the preselected criterion and the cell culture or process bioreactor sample being evaluated, the cell culture sample or process bioreactor sample being evaluated is obtained at a different time than when the culture cell sample or process bioreactor sample is obtained for the preselected criterion. For example, the cell culture sample or process bioreactor sample can be obtained 1, 2, 3, 5, 10, 15, 30, 50, 70, 100, 200, 300 days, 1 year, 2 years or more after the cell culture sample or bioreactor sample is obtained that provided the preselected criterion.

In one embodiment, the element of the process for producing a polypeptide preparation that is being evaluated is a culture process. In one embodiment, the culture process is one or more of pH, feeding conditions, osmolarity, carbon dioxide levels, shear force or agitation rate, temperature, oxidation, cell density, seeding density, timing and sparge rate. In one embodiment, the culture process is a batch culture, a fed-batch culture, a perfusion culture or a continuous culture.

In one embodiment, the element of the process for producing a polypeptide preparation that is being evaluated is a media component. The media component can be one or more of: buffer, amino acid content, vitamin content, salt content, mineral content, carbon source content, lipid content, nucleic acid content, hormone content, trace element content, ammonia content, surfactant content, sugar content, sugar precursor content, enzyme content, indicator content, and small molecule content.

In one embodiment, the process characteristic is the absence, presence or amount of a vitamin or vitamins in the cell culture sample or the process bioreactor sample. In such embodiments, the method evaluates the absence, presence, value or amount of a vitamin or vitamins in a cell culture sample or process bioreactor sample. Examples of vitamins that can be evaluated include: vitamin A (retinoid), vitamin B1 (thiamine), vitamin B2 (riboflavin), vitamin B3 (niacin), nicotinamide, vitamin B5 (pantothenic acid), vitamin B6 (pyrodoxal, pyridoxine, pyridoxamine), vitamine B7 (biotin), vitamin B9 (folic acid), vitamin B12 (cyanocobalamin), vitamin C (ascorbic acid), vitamin D, vitamin E, and vitamin K. In some embodiments, when the process characteristic is the absence, presence or amount of vitamin B7 content or calcium pantothenate content, the media is uninoculated cell culture media (e.g., reconstituted dry powder media). In one embodiment, the liquid chromatography is ultra high performance liquid chromatography, e.g., ultra high performance liquid chromatography that includes a sub-2 micrometer particle comprising a C1 to C18 ligand capable of separating highly polar compounds, e.g., a high strength silica core.

In one embodiment, the process characteristic is the absence, presence, value or amount of the polypeptide or a polypeptide isoform. In such embodiments, the method evaluates the absence, presence, value or amount of a polypeptide or polypeptide isoform in a cell culture sample or process bioreactor sample. In one embodiment, the liquid chromatography (LC) is reverse phase LC, size exclusion LC, normal phase LC, hydrophobic interaction LC, anion exchange LC, or weak cation exchange LC. In some embodiments, the LC is high performance LC (HPLC), e.g., HPLC of the aforementioned LC methods.

In one embodiment, the polypeptide is a polypeptide isoform having a particular glycan structure, e.g., a glycan structure described herein. An isoform having, e.g., one or more of the following glycan structures can be evaluated a complex glycan, a high mannose glycan and a hybrid glycan. In one embodiment, the polypeptide isoform can have one or more of the complex glycan, high mannose glycan and hybrid glycan structures described herein.

In one embodiment, the process characteristic being evaluated can be a polypeptide isoform that has a modified amino acid sequence of the polypeptide or that has a linkage structure that differs from the polypeptide. In one embodiment, the polypeptide isoform has a modification of the amino acid sequence of the polypeptide and the modification is one or more of methionine oxidation, asparginine deamination, aspartic acid isomerization, and cysteinylation of one or more amino acids encoding the polypeptide. In another embodiment, the polypeptide isoform has one or more amino acids in addition to the amino acid sequence of the polypeptide. For example, the polypeptide isoform can have an N-terminal amino acid extension as compared to the amino acid sequence of the polypeptide, e.g., the polypeptide is an antibody and the antibody isoform has an N-terminal amino acid extension of the light chain of the antibody. In other embodiments, the polypeptide isoform has one or more amino acids less than the amino acid sequence of the polypeptide, e.g., the polypeptide is an antibody and the antibody isoform has one or more amino acids removed from the hinge region of the antibody and/or the antibody isoform has a lysine residue removed from the C-terminus of a heavy chain of the antibody. In one embodiment, the polypeptide is an antibody and the antibody isoform is a light chain or a light chain multimer (e.g., a light chain dimer). For example, the antibody isoform can be a light chain isomer that is cysteinylated, a light chain isomer that is glutathionylated, and/or a light chain isomer that has a free reduced sulfohydryl group. In one embodiment, the polypeptide is an antibody and the antibody isoform is a multimer containing combinations of light chain and heavy chain including a dimmer of light chain and heavy chain and a trimer containing two heavy chains and a light chain. For example, the antibody isoform can be a heavy chain isomer that is cysteinylated, a heavy chain isomer that is glutathionylated, and/or a heavy chain isomer that has a free reduced sulfohydryl.

In one embodiment, the process characteristic being evaluated can be a polypeptide isoform that differs from the polypeptide by one or more post translational modification, e.g., one or more post translational modification described herein. Exemplary post translational modifications include proteolysis, racemization, N—O acyl shift, multimerization, aggregation, glycosylation, cysteinylation, neddylation, acylation, formylation, myristoylation, pyroglutamate formation, methylation, glycation, carbamoylation, amidation, glycosyl phosphatidylinositol addition, O-methylation, glypiation, ubiquitination, SUMOylation, methylation, acetylation, hydroxylation, ubiquitination, desmosine formation, deamination and/or oxidation, imine formation, disulfide bond formation, prenylation, palmitoylation, phosphorylation, dephosphorylation, sulfation, porphyrin ring linkage, flavin linkage, GFP prosthetic group (Thr-Tyr-Gly sequence) formation, lysine tyrosine quinone (LTQ) formation, topaquinone (TPQ) formation, succinimide formation, transglutamination, carboxylation, polyglutamylation, polyglycylation, and citrullination.

In one embodiment, the method further includes subjecting the culture sample or process bioreactor sample to a UV detector, fluorescence detector, refractive index detector or mass detector, e.g., a mass detector capable of scanning to 4000 m/z or greater, to determine the structure of the process characteristic. In one embodiment, the structure of the process characteristic is determined using mass spectrometry. Examples of mass spectrometry that can be used include one or more of electrospray ionization mass spectrometry (ESI-MS), turbospray ionization mass spectrometry, nanospray ionization mass s spectrometry, thermospray ionization mass spectrometry, sonic spray ionization mass spectrometry, surface enhanced laser desorption ionization mass spectrometry (SELDI-MS), adjacent power interference mass spectrometry (APCI-MS) and matrix assisted laser desorption/ionization mass spectrometry (MALDI-MS).

In preferred embodiments, the test value can be memorialized, e.g., in a computer readable record. The preselected criterion can be, e.g., a release specification, a regulatory required specification, e.g., a process analytical technology (PAT) requirement or a label requirement. In some embodiments, the preselected criterion is a different preparation of the polypeptide, e.g., a reference standard prepared by a different process than the polypeptide preparation being evaluated. For example, the reference standard can be prepared by a different manufacturing process, e.g., culture and/or isolation step, or different expression system. In other embodiments, a reference standard is prepared by the same process as the polypeptide preparation being evaluated. For example, the reference standard can be prepared by the same manufacturing process, e.g., the same culture, isolation and expression system, as the polypeptide preparation being evaluated, e.g., the reference standard can be determined at a different time point during the manufacture process than the polypeptide preparation being evaluated, e.g., to evaluate changes in the output of the manufacture process over time.

In one embodiment, the method further includes making a decision about the manufacturing process based upon the analysis. The decision can be to maintain the manufacturing process based, at least in part, upon the analysis or to change one or more elements of the manufacturing process, e.g., to change one or more of a cell or cell line used to produce the polypeptide preparation, an element of a vector used to transform or transfect a cell (or progeny of the cell) that produces the polypeptide preparation, a component of the culture media, a culture process, a purification process, and a formulation process, based at least in part upon the analysis.

In some embodiments, the method further includes making a decision about a polypeptide preparation prepared the manufacturing process being evaluated. The decision can be, e.g., one or more of accepting or discarding the polypeptide preparation, releasing or withholding the polypeptide preparation, formulating the polypeptide preparation, packaging the polypeptide preparation, labeling the polypeptide preparation, shipping, relocating, selling or offering to sell the polypeptide preparation.

In another aspect, the disclosure features a method of evaluating a process of producing a polypeptide preparation, or an element of a process used to produce a polypeptide preparation, the method comprises: providing a determination about the process or an element of the process based upon a method described herein. The determination can be to maintain the process based, at least in part, upon the analysis or to change one or more elements of the process, e.g., to change one or more of a cell or cell line used to produce the polypeptide preparation, an element of a vector used to transform or transfect a cell (or progeny of the cell) that produces the polypeptide preparation, a component of the culture media, a culture process, a purification process, and a formulation process, based at least in part upon the analysis.

Methods disclosed herein are useful for analyzing or processing a polypeptide preparation, e.g., to guide the control of a step or steps in the production of a polypeptide preparation.

In another aspect, the disclosure features a database that can include one or more of a preselected criterion and a test value, as determined by a method described herein.

In one embodiment the database is: disposed on tangible medium; disposed on a single unit of tangible medium, e.g., on a single computer, or in a single paper document; provided on more than one unit of tangible medium, e.g., on more than one computer, in more than a single paper document, partly on a paper document and partly on computer readable medium; disposed on computer readable medium; disposed on traditional medium, e.g., paper, which is readable by a human without the use of a computer, e.g., a printed document, chart, table or card catalogue.

Methods described herein allow for rapid analysis of polypeptide production during the manufacturing process because the methods can be applied to cell culture samples and process bioreactor samples without first subjecting the sample to affinity chromatography. The methods described herein are also capable of detecting trace isoforms of a polypeptide and other biomolecules present in the sample at concentrations as low as about 0.007 μg/μL. For manufacturing process development, the methods described herein allow for the direct analysis of polypeptides and biomolecules in cell culture medium during the production of recombinant proteins. The methods have a number of applications including: on- or off-line polypeptide concentration determination, cell line development, clone selection, cell line optimization, optimization of protein primary sequence and vector construction, bioreactor process optimization, rapid comparison of bioreactor process changes, new technology development of manufacturing processes, and process analytical technologies (PAT). In addition, the methods described herein can be used as a characterization tool during early process development can provide insights for downstream processing and the development of additional and specific analytical assays.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A is an overlay of five chromatograms for mAb-1 at different HPLC elution conditions showing from left to right a linear gradient of 3% B/min, 2% B/min, 1.5% B/min, 1% B/min, and 0.75% B/min over the analytical range of 17% B-42% B for a fixed flow rate of 100 μL/min.

FIG. 1B is an overlay of five chromatograms for mAb-2 at different HPLC elution conditions showing from left to right a flow rate of 400 μL/min, 300 μL/min, 200 μL/min, 150 μL/min, and 100 μL/min.

FIG. 2 depicts four chromatograms for mAb-1 at different heated column temperatures 35° C., 45° C., 55° C., and 65° C. from top to bottom showing at left, full scale chromatograms with a fixed maximum of 2.00 AU on the UV absorbance Y-axis, and at right, zoomed-in chromatograms with a fixed maximum of 0.07 AU on the UV absorbance Y-axis, for 15 μL of 0.20 μg/μL mAb injected on a column with measured absorbance at 214 nm.

FIG. 3A panels are chromatograms of mAb-1, mAb-2, and mAb-3 DS from top to bottom by Poroshell 300SB-C3 RP-HPLC detected at 214 nm for 25 μL of 0.20 μg/μL mAb DS injected for a total of 5 μg of intact mAb on column.

FIG. 3B panels are deconvoluted, zero-charge mass spectra from top to bottom of the detected chromatographic peak for mAb-1, mAb-2, and mAb-3 DS as shown in FIG. 1A using MaxEnt-1 calculations: 1.000 Da/Channel (Resolution), 0.750 Da (Uniform Gaussian Width), and 67% (Minimum intensity ratio).

FIG. 4 is a graph of peak area response, at 214 nm, on the y-axis as a function of amount of antibody on column (μg) on the x-axis for mAb-1 and mAb-3 over a range of 1 μg (6.8 μmol) to 5 μg (34 μmol) as determined by Poroshell 300SB-C3 RP-HPLC/UV.

FIG. 5 panels are three chromatograms of conditioned bioreactor process samples from an intermediate-scale bioreactor for mAb-1 (top panel), mAb-2 (middle panel), and mAb-3 (bottom panel) showing peak identities as determined by on-line ESI-QTOF MS analysis of 15 μL of 10-fold diluted conditioned bioreactor process sample (diluted with 0.05% TFA in water) injected on column.

FIG. 6A panels are chromatograms of 20 μL of a 10-fold dilution (in 0.05% TFA in water) of conditioned bioreactor process samples taken at distinct bioreactor time points during the last half of the cell culture manufacturing process of mAb-1.

FIG. 6B panels are chromatograms of 15 μL of a 10-fold dilution (in 0.05% TFA in water) of conditioned bioreactor process samples taken at distinct bioreactor time points during the last half of the cell culture manufacturing process of mAb-3.

FIG. 7A panels are RP-HPLC/UV chromatogram measured at 214 nm of 15 μL of 10-fold dilution (in 0.05% TFA in water) of conditioned bioreactor process samples of mAb-3 from four different lab-scale bioreactor processes by 300SB-C3 RP-HPLC/UV/ESI-QTOF MS.

FIG. 7B panels are deconvoluted, zero-charge mass spectra for the free light chain peak observed for each process described in FIG. 7A using MaxEnt-1 calculations: 1.000 Da/Channel (Resolution), 0.900 Da (Uniform Gaussian Width), and 67% (Minimum intensity ratio) showing observed masses that agree with the theoretical masses of 22,812.1 Da and 22,998.3 Da for light chain with cysteinylation and glutathionylation respectively.

FIG. 7C panels are deconvoluted, zero-charge mass spectra for the intact mAb peak observed for each process described in FIG. 7A using MaxEnt-1 calculations: 1.000 Da/Channel (Resolution), 0.900 Da (Uniform Gaussian Width), and 67% (Minimum intensity ratio) showing observed masses that agree with the theoretical masses of 147,370.3 Da and 147,532.4 Da for intact mAb with G0F/G0F and G0F/G1F N-linked glycosylation respectively.

FIG. 8 panels are representative chromatograms measured at 445 nm for B12 and Riboflavin Standard Vitamin Mix (top panel), sample taken at day 2 (middle panel), and sample taken at day 10 (bottom panel).

FIG. 9 panels are representative chromatograms measured at 345 nm for Folic Acid Standard Vitamin Mix (top panel), sample taken at day 2 (middle panel), and sample taken at day 10 (bottom panel).

FIG. 10 panels are representative chromatograms measured at 326 nm for Pyridoxal and Pyridoxine Standard Vitamin Mix (top panel), sample taken at day 2 (middle panel), and sample taken at day 10 (bottom panel).

FIG. 11 panels are representative chromatograms measured at 262 nm for Nicotinamide and Thiamine Standard Vitamin Mix (top panel), sample taken at day 2 (middle panel), and sample taken at day 10 (bottom panel).

FIG. 12 panels are representative chromatograms measured at 205 nm for Calcium Pantothenate and Biotin Standard Vitamin Mix (top panel), uninoculated media (middle panel), and sample taken at day 10 (bottom panel).

### DETAILED DESCRIPTION OF THE INVENTION

Methods described herein provide analytical techniques for evaluating a process characteristic of cell culture samples and process bioreactor samples used in the production of polypeptide preparations, e.g., recombinant polypeptide preparations. For example, methods disclosed herein can be used to evaluate a process for the preparation of an antibody or antigen-binding fragment thereof. The use of liquid chromatography, e.g., HPLC, allows for the analysis of cell culture and process bioreactor samples that have not been subjected to chromatography, e.g., affinity chromatography, prior to the analysis.

The term “cell culture medium” refers to a solution containing nutrients to support cell survival under conditions in which cells can grow and produce a desired protein. A person of ordinary skill in the cell culture art will know without undue experimentation what components make-up cell culture media. Typically, these solutions provide essential and non-essential amino acids, vitamins, energy sources, lipids, and trace elements required by a cell for growth and survival.

The term “cell-free conditioned medium” as used herein refers to the supernatant that is generated from the removal of cells and cellular debris by a separation method, such as centrifugation and/or microfiltration, from cell culture medium that has been exposed to host cells, which may secrete desired recombinant polypeptide(s) of interest. Cell-free conditioned medium can contain the secreted recombinant polypeptide, or product, of interest; selected nutrients (e.g. vitamins, amino acids, cofactors, and minerals); additional growth factors/supplements including insulin; and additional exogenous, or host cell, proteins.

The term “protein” as used herein refers to one or more polypeptides that can function as a unit. The term “polypeptide” as used herein refers a sequential chain of amino acids linked together via peptide bonds. The term “polypeptide” is used to refer to an amino acid chain of any length, but one of ordinary skill in the art will understand that the term is not limited to lengthy chains. If a single polypeptide can function as a unit, the terms “polypeptide” and “protein” may be used interchangeably. A “polypeptide” refers to the amino acid chain. An “isoform” of a polypeptide, as used herein, refers to version of a polypeptide with small differences to another isoform of the same polypeptide. Different forms of a polypeptide can result from transcription or translation of the sequence encoding the polypeptide, as well as differences arising from the processing and secretion of the polypeptide from a cell, from purification, from formulation and from degradation during storage. An isoform can vary from the polypeptide by differences in amino acid sequence, multimerization, glycosylation and other post translational modifications such as those described herein. A “glycoform” is an isoform where different versions of a glycoprotein have different polysaccharides attached to them, by posttranslational modifications.

The term “antibody” refers to any immunoglobulin. Fragment of antibodies are also included in the methods of the invention along with any peptide or polypeptide comprising an antigen-binding site. Methods of the invention include, but are not limited to the use of, polyclonal, monoclonal, mono-specific, poly-specific, bi-specific, humanized, de-immunized, human, camelid, rodent, single-chain, chimeric, synthetic, recombinant, hybrid, mutated, grafted, and in vitro generated antibodies. Also included are antibody fragments and variant molecules such as Fab, F(ab′)₂, Fv, scFv, Fd, dAb, VHH, and other antibody fragments and variant molecules that retain antigen-binding function. Typically, such fragments would comprise an antigen-binding domain.

The term “preparation” refers to any composition containing at least one polypeptide.

The term “therapeutic protein” refers to a protein or peptide that has a biological effect on a region in the body on which it acts or on a region of the body on which it remotely acts via intermediates. A therapeutic protein can be, for example, a secreted protein, such as, an antibody, an antigen-binding fragment of an antibody, a soluble receptor, a receptor fusion, a cytokine, a growth factor, an enzyme, or a clotting factor, as described in more detail herein below. The above list of proteins is merely exemplary in nature, and is not intended to be a limiting recitation. One of ordinary skill in the art will understand that any protein may be used in accordance with the present invention and will be able to select the particular protein to be produced based as needed.

Methods of analyzing a production process or an element of a production process are described below.

### Process Characteristics

Methods disclosed herein provide for analysis of a process characteristic or characteristics of a polypeptide preparation prepared under certain production parameters. The analysis can be, e.g., the absence, presence, value or amount of a process characteristic. A “process characteristic” as used herein refers to a property of a cell culture sample or process bioreactor sample that can be effected by one or more production parameters used in a method of producing a polypeptide preparation in cell culture or a bioreactor. A process characteristic can be the polypeptide being produced, an isoform of polypeptide being produced, other proteins present during the production of a polypeptide of interest and other media components present during the production of a polypeptide such as vitamins.

The process characteristic can be a post translational modification of the polypeptide. Examples of post-translational modifications that can be included are: proteolysis, racemization, N—O acyl shift, multimerization, aggregation, glycosylation, cysteinylation, biotinylation, neddylation, acylation, formylation, myristoylation, pyroglutamate formation, methylation, glycation, carbamoylation, amidation, glycosyl phosphatidylinositol addition, O-methylation, glypiation, ubiquitination, SUMOylation, methylation, acetylation, hydroxylation, ubiquitination, desmosine formation, deamination and/or oxidation, imine formation, disulfide bond formation, prenylation, palmitoylation, phosphorylation, dephosphorylation, sulfation, porphyrin ring linkage, flavin linkage, GFP prosthetic group (Thr-Tyr-Gly sequence) formation, lysine tyrosine quinone (LTQ) formation, topaquinone (TPQ) formation, succinimide formation, transglutamination, carboxylation, polyglutamylation, polyglycylation, and citrullination.

Specific examples of posttranslational modifications include: C-terminus modifications (e.g., a C-terminal amide), N-terminus modifications (e.g., N-terminal methylation, aceylation, glycation, biotinylation and carbalylation), cysteine modifications (e.g., disulfide bond reduction, disulfide bond formation, carboxymethyl cysteine, carboxyamidomethyl cysteine, pyridylethyl cysteine, NEM cysteine and methionine overalkylation (IodoAc, IodoAM)), amino acid modifications (e.g., lysine methylation, lysine dimethylation, arganine dimethylation, lysine trimethylation, lysine acetylation, aspartic acid hydroxylation, proline hydroxylation, tryptophan hydroxylation, aspartic acid carboxylation, glutamic acid carboxylation, tyrosine sulfation, serine phosphorylation, threonine phosphorylation, tyrosine phosphorylation, lysine biotinylation, cyesteinylation, histidine oxidation, methionine oxidation, tryptophan oxidation, asparganine deamination, succinimide from asparganine, succinimide from aspartic acid, pyroglutamic acid from glutamic acid, pyroglutamic acid from glutamine, dehydroalanine from serine, dehydrothreonine, cysteine oxidation (+1, +2, +3), dehydroalanine cysteine, S-carbamoylmethyl cysteine, lysine glycation), and amino acid substitutions (e.g., glutamine to lysine, lysine to glutamine, asparganine to aspartate, aspirate to asparganine, glutamine to glutamate, isoleucine to asparganine, asparganine to isoleucine, lysine to glutamate, glutamate to lysine, lysine to methionine, methionine to lysine, praline to threonine, threonine to praline, glutamine to histidine, histidine to glutamine, serine to praline, praline to serine, threonine to isoleucine, isoleucine to threonine, threonine to asparganine, asparganine to threonine, asparganine to lysine, lysine to asparganine, aspartate to glutamate, glutamate to aspartate, glycine to alanine, alanine to glycine, serine to threonine, threonine to serine, valine to isoleucine, isoleucine to valine, valine to lysine, lysine to valine, isoleucine to lysine, lysine to isoleucine, leucine to valine, valine to leucine, isoleucine to lysine, lysine to isoleucine, leucine to glutamine, glutamine to leucine, alanine to serine, serine to alanine, phenylalanine to alanine, phenylalanine to tyrosine, tyrosine to phenylalanine, praline to leucine, leucine to praline, serine to cysteine, cysteine to serine, valine to aspartate, aspartate to valine, isoleucine to methionine, methionine to isoleucine, leucine to methionine, methionine to leucine, methionine to Nle, histidine to arganine, arganine to histidine, aspartate to histidine, histidine to aspartate, asparganine to histidine, histidine to asparganine, leucine to histidine, histidine to leucine, methionine to arganine, arganine to methionine, alanine to praline, praline to alanine, histidine to tyrosine, tyrosine to histidine, serine to isoleucine, isoleucine to serine, serine to leucine, leucine to serine, serine to asparganine, asparganine to serine, threonine to lysine, lysine to threonine, alanine to valine, valine to alanine, glutamine to arganine, lysine to arganine, arganine to lysine, alanine to threonine, threonine to alanine, arganine to tryptophan, tryptophan to arganine, glycine to serine, serine to glycine, threonine to methionine, methionine to threonine, valine to glutamate, glutamate to valine, praline to glutamine, glutamine to praline, valine to methionine, methionine to valine, isoleucine to phenylalanine, phenylalanine to isoleucine, leucine to phenylalanine, phenylalanine to histidine, histidine to phenylalanine, glycine to valine, valine to glycine, isoleucine to arganine, arganine to isoleucine, leucine to arganine, arganine to leucine, alanine to aspartate, aspartate to alanine, cysteine to phenylalanine, phenylalanine to cysteine, glycine to cysteine, cysteine to glycine, aspartate to tyrosine, tyrosine to aspartate, valine to phenylalanine, phenylalanine to valine, asparganine to tyrosine, tyrosine to asparganine, cysteine to arganine, arganine to cysteine, threonine to arganine, arganine to threonine, alanine to glutamate, glutamate to alanine, glycine to aspartate, aspartate to glycine, praline to arganine, arganine to praline, cysteine to tyrosine, tyrosine to cysteine, serine to phenylalanine, phenylalanine to cysteine, serine to phenylalanine, phenylalanine to serine, serine to arganine, arganine to serine, glycine to glutamate, glutamate to glycine, leucine to tryptophan, tryptophan to leucine, serine to tyrosine, tyrosine to serine, cysteine to tryptophan, tryptophan to cysteine, glycine to arganine, arganine to glycine, serine to tryptophan, tryptophan to serine, glycine to tryptophan and tryptophan to glycine).

In some embodiments, the process characteristic to be analyzed can be the absence, presence or amount of a glycoform of the polypeptide. Proteins and polypeptides can be glycosylated at arginine residues, referred to as N-linked glycosylation or N-linked glycans, and at serine or threonine residues, referred to as O-linked glycosylation or O-linked glycans. Exemplary N-linked linked glycans include complex, high mannose and hybrid glycans. Complex, high mannose and hybrid glycans can result from different processing events to a precursor oligosaccharide that can occur in a cell. A “precursor oligosaccharide” as used herein refers to the oligosaccharide chain involved in the initial steps in biosynthesis of carbohydrate chains. A “precursor oligosaccharide” can be an oligosaccharide structure which includes at least the following sugars: Man₉GlcNAc₂, for example, a precursor oligosaccharide can have the following structure: Glc₃Man₉GlcNAc₂. High mannose glycans can include three to nine mannose residues attached to GlcNAc₂. Exemplary high mannose glycans that can be evaluated by the methods described herein include: a high mannose glycan having five mannose residues (also referred to as “Man5”), a high mannose glycan having six mannose residues (also referred to as “Man6”), a high mannose glycan having seven mannose residues (also referred to as “Man7”) and D1, D2 and D3 isomers thereof, a high mannose glycan having eight mannose residues (also referred to as “Man 8”) and D1 D2, D2D3, and D1D3 isomers thereof, and a high mannose glycan having nine mannose residues (also referred to as “Man9”). High mannose glycans can further include a glucose residue (e.g., Man7+Glc, Man8+Glc, Man9+Glc).

Complex glycans include a pentasaccharide core of Man₃GlcNAc₂, which can be fucosylated or non-fucosylated, and may contain one, two, three or four outer branches (“antennae”) attached to pentasaccharide core. These structures are referred to in terms of the number of their outer branches: monoantennary (one branch), biantennary (two branches), triantennary (three branches) or tetraantennary (four branches). Exemplary complex glycans that can evaluated by the methods disclosed herein include: a fucosylated biantennary complex glycan having no reducing end terminal galactose residues (also referred to herein as “G0F”), a fucosylated biantennary complex glycan having one reducing end terminal galactose residue (also referred to herein as “G1F”), a fucosylated biantennary complex glycan having two reducing end terminal galactose residues (also referred to as “G2F”), a non-fucosylated biantennary complex glycan having no reducing end terminal galactose residues (also referred to herein as “G0”), a non-fucosylated biantennary complex glycan having one reducing end terminal galactose residue (also referred to herein as “G1”), a non-fucosylated biantennary complex glycan having two reducing end terminal galactose residues (also referred to herein as “G2”), a fucosylated biantennary complex glycan having two galactose residues and one N-acetylneuraminic acid residue (also referred to herein as “G2F/NeuAc”), a fucosylated biantennary complex glycan having two galactose residues and two N-acetylneuraminic acid residues (also referred to as “G2F/2NeuAc”) and any of these structures having one or two terminal sialic acid residues. Other complex glycan structures that can be evaluated include monoantennery structures (e.g., (GlcNAc) Man₃ GlcNAc₂; (Gal-GlcNAc) Man₃ GlcNAc₂; (NeuAc-Gal-GlcNAc) Man₃ GlcNAc₂; (GlcNAc) Man₃ GlcNAc₂ (Fuc); (Gal-GlcNAc) Man₃ GlcNAc₂ (Fuc); and (NeuAc-Gal-GlcNAc) Man₃ GlcNAc₂ (Fuc). Exemplary triantennary complex glycans that can be evaluated include: G0, G0F (there are two different Tri-antennary linkage configurations, Tri and Tri′); G1 (there are numerous isomeric configurations for all G1 and G2 species beyond Tri and Tri′); G1+1 NeuAc; G1+1 NeuGc; G1F; G1F+1 NeuAc; G1F+1 NeuGc; G2; G2+1 NeuAc; G2+1 NeuGc; G2+2 NeuAc; G2+2 NeuGc; G2F; G2F+1 NeuAc; G2F+1 NeuGc; G2F+2 NeuAc; G2F+2 NeuGc; G3 (there are numerous isomeric configurations for the G3 sialylated species beyond Tri and Tri′); G3+1 NeuAc; G3+1 NeuGc; G3+2 NeuAc; G3+2 NeuGc; G3+3 NeuAc; G3+3 NeuGc; G3F; G3F+1 NeuAc; G3F+1 NeuGc; G3F+2 NeuAc; G3F+2 NeuGc; G3F+3 NeuAc; G3F+3 NeuGc; G3F+3 NeuAc+1 N-acetyllactosamine; G3F+3 NeuGc+1 N-acetyllactosamine; G3F+3 NeuAc+2 N-acetyllactosamine; and G3F+3 NeuGc+2 N-acetyllactosamine. Exemplary tetrantennary complex glycan structures that can be evaluated by the methods described herein include: G0; G0F; G1 (there are numerous isomeric configurations for all G1, G2 and G3 species); G1+1 NeuAc; G1+1 NeuGc; G1F; G1F+1 NeuAc; G1F+1 NeuGc; G2; G2+1 NeuAc; G2+1 NeuGc; G2+2 NeuAc; G2+2 NeuGc; G2F; G2F+1 NeuAc; G2F+1 NeuGc; G2F+2 NeuAc; G2F+2 NeuGc; G3; G3+1 NeuAc; G3+1 NeuGc; G3+2 NeuAc; G3+2 NeuGc; G3+3 NeuAc; G3+3 NeuGc; G3F; G3F+1 NeuAc; G3F+1 NeuGc; G3F+2 NeuAc; G3F+2 NeuGc; G3F+3 NeuAc; G3F+3 NeuGc; G4; G4+1 NeuAc (there are numerous isomeric configurations for the G4 sialylated species); G4+1 NeuGc; G4+2 NeuAc; G4+2 NeuGc; G4+3 NeuAc; G4+3 NeuGc; G4+4 NeuAc; G4+4 NeuGc; G4F; G4F+1 NeuAc; G4F+1 NeuGc; G4F+2 NeuAc; G4F+2 NeuGc; G4F+3 NeuAc; G4F+3 NeuGc; G4F+4 NeuAc; G4F+4 NeuGc; G4F+4 NeuAc+1 N-acetyllactosamine; G4F+4 NeuGc+1 N-acetyllactosamine; G4F+4 NeuAc+2 N-acetyllactosamine; G4F+4 NeuGc+2 N-acetyllactosamine, G4F+4 NeuAc+3 N-acetyllactosamine, and G4F+4 NeuGc+3 N-acetyllactosamine. O-linked glycan structures include, but are not limited to: Core 1 structures (GalNAc; GalNAc-Gal; GalNAc-Gal-NeuAc; and GalNAc(NeuAc)-Gal-NeuAc); Core 2 structures (GalNAc(GlcNAc)-Gal; GalNAc(GlcNAc)-Gal-NeuAc; GalNAc(GlcNAc-Gal)-Gal-NeuAc; GalNAc(GlcNAc-Gal-NeuAc)-Gal-NeuAc; and GalNAc(GlcNAc[Fuc]-Gal-NeuAc)-Gal-NeuAc) and unique structures (GlcNAc; Glc-Xyl; Glc-Xyl-Xyl; Fuc-GlcNAc-Gal-NeuAc; and Fuc). In addition, isomers of complex glycans having a monosaccharide residue present on one chain but not on another can be analyzed by the methods described herein.

Hybrid glycans can include one or more high mannose branch and one or more complex glycan branch. Again, isomers of a hybrid glycan can also be analyzed by methods disclosed herein. Exemplary hybrid glycans include: 2 Man (GlcNAc) Man₃ GlcNAc₂ (Fuc); 2 Man (Gal-GlcNAc) Man₃ GlcNAc₂ (Fuc); 2 Man (NeuAc-Gal-GlcNAc) Man₃ GlcNAc₂ (Fuc); 1 Man (GlcNAc) Man₃ GlcNAc₂ (Fuc); 1 Man (Gal-GlcNAc) Man₃ GlcNAc₂ (Fuc); 1 Man (NeuAc-Gal-GlcNAc) Man₃ GlcNAc₂ (Fuc); 2 Man (GlcNAc) Man₃ GlcNAc₂; 2 Man (Gal-GlcNAc) Man₃ GlcNAc₂; 2 Man (NeuAc-Gal-GlcNAc) Man₃ GlcNAc₂; 1 Man (GlcNAc) Man₃ GlcNAc₂; 1 Man (Gal-GlcNAc) Man₃ GlcNAc₂; and 1 Man (NeuAc-Gal-GlcNAc) Man₃ GlcNAc₂.

In some embodiments, the polypeptide is an antibody, or antigen binding fragment thereof, and the process characteristic being analyzed can be one or more of: methionine oxidation, fragmentation of the hinge region, processing of a C-terminal lysine on the heavy chain of the antibody, linkages (e.g., incomplete linkages or free sulfohydryl groups), aggregation, multimerization (e.g., intact antibody or light chain multimers) or lack of multimerization (e.g., free light chain), asparganine deamination, aspartic acid isomerization, N-terminal pyroglutamic acid, N-terminal amino acid extensions of the light chain (e.g., due to alternative signal cleavage), cysteinylation, glutathionylated, and glycan structure. Other antibody modifications including other modifications described herein are known in the art and can be evaluated by the methods described herein.

Post translational modifications associated, e.g., with production in Chinese hamster ovary (CHO) cells include decarboxylation of gamma carboxy glutamate, amide formation at the C-terminus, methylation (e.g., at the N-terminus, N epsilon of lysine, O of serine, threonine or the C-terminus, and N of asparganine); hydroxylation (e.g., of delta C of lysine, beta C of tryptophan, C3 or C4 of proline, beta C of aspartate); N,N dimethylation (e.g., of arganine or lysine); ethylation, formylation, acetylation (e.g., N-terminus, N epsilon of lysine, O of serine); trimethylation (e.g., of lysine); carboxylation (e.g., of aspartate or glutamate); sulfation (e.g., of O of tyrosine and O of serine); phosphorylation (e.g., O of serine, threonine, tyrosine and aspartate, N epsilon of lysine); C-mannosylation of tryptophan; laurylation; farnesylation; myriosylation; biotinylation; palmitoylation; stearoylation and geranylgeranylation. Cysteine-related modifications include: underalkylation of cysteine (e.g., after reduction/alkylation with iodoacetic acid or iodoacetamide); lysinoalanine; lanthionine; dehydroalanine; disulfide bond formation; sulfenic acid; sulfinic acid; cysteic acid, carboxamidomethyl of cysteine; carboxymethyl on cysteaine; pyridylethylation of cysteine and glutathionylation. Methione related modifications include: homoserine formed from methionine by CNBr treatment; misincorporation of norleucine for methionine; overalkylation of methionine (after reduction/alkylation with iodoacetic acid); oxidation of methionine to sulphoxide; and oxidation of methionine to sulphone. One or more of these post translational modifications can be a process characteristic of a polypeptide evaluated by the methods described herein.

Post translational modifications that can occur during manufacturing and storage of polypeptide preparations, e.g., prepared using CHO cells, include: pyroglutamic acid formed from glutamic acid; dehydration (—H₂O); serine to dehydroalanine; succinimide formation from aspartic acid; pyroglutamic acid formed from glutamine; succinimide formation from asparagine; S-carbamoylmethylcysteine cyclization (N-terminus); deamidation of asparagine and glutamine to aspartate and glutamate; oxohistidine (from histidine); single oxidation of tryptophan; formylation; double oxidation of tryptophan; acetylation (N terminus, N epsilon of Lysine, O of Serine) (Ac); carbamoylation and glycation of lysine and N-terminus.

A process characteristic can also be a media component. For example, methods described herein can be used to determine the presence, absence or amount of a process related polypeptide and/or vitamin. Examples of vitamins that can be evaluated from a cell culture sample or process bioreactor sample include: vitamin A (retinoid), vitamin B1 (thiamine), vitamin B2 (riboflavin), vitamin B3 (niacin), nicotinamide, vitamin B5 (pantothenic acid), vitamin B6 (pyrodoxal, pyridoxine, pyridoxamine), vitamine B7 (biotin), vitamin B9 (folic acid), vitamin B12 (cyanocobalamin), vitamin C (ascorbic acid), vitamin D, vitamin E, and vitamin K. When vitamin B7 content or calcium pantothenate content is being evaluated, the media is preferably uninoculated cell culture media (e.g., reconstituted dry powder media).

### Liquid Chromatography

Liquid chromatography (LC) can be used to analyze, e.g., biomolecules, polypeptides, small molecules, and vitamins in a cell culture medium or a bioreactor process sample. Reversed-phase LC is one type of LC that can be used to study the process characteristics described herein. In one embodiment, ultra high performance liquid chromatography (UHPLC) is used. In another embodiment, reverse-phase UHPLC is used to analyze a process characteristic in cell culture media. For some embodiments, e.g., polypeptide analysis, other LC forms, e.g., reverse-phase LC, size exclusion LC, normal phase LC, hydrophobic interaction LC, anion exchange LC, weak cation exchange LC, and similar chromatographic techniques can also be used. In certain embodiments, the aforementioned LC forms are a high performance LC. For some applications, e.g., analysis of vitamins, any ultra-high performance liquid chromatography type instrument that has similar capabilities can be used. One system that can be used is the ACQUITY UPLC® chromatography system (Waters Corp., Milford Mass.). In one embodiment, UHPLC is used for analysis of vitamin process characteristics.

The HPLC process can be interfaced with automated sampling technologies that can enable on-line or at-line monitoring of the sample with real-time (or near real-time) analysis. The sample to be analyzed can be obtained from a bioreactor, e.g., lab-scale, intermediate-scale, and full-scale bioreactor. The sample can be a cell culture medium, and in some embodiments, the sample is a conditioned medium (i.e., centrifuged to remove substantially all or all cells) or an uninoculated cell culture medium. The sample can include recombinant proteins, e.g., antibodies, e.g., monoclonal antibodies, process-related impurities and byproducts, e.g., host cell proteins, and vitamins. The sample can be directly analyzed by HPLC, without a chromatographic preparation step, such as affinity chromatography. In some applications, any cells present in the sample can be removed, e.g., by centrifugation and/or filtering (through, e.g., a 0.22 μm filter). Samples can be injected on the column either neat, acidified, or diluted with an appropriate solvent solution. Suitable internal standards are used.

Various types of substrates can be used with the HPLC columns or UPLC columns, e.g., silica. For some applications, e.g., protein analysis, superficially-porous silica particles (“Poroshell”) with solid cores and thin, uniformly porous, outer shells (see, e.g., Kirkland et al., J. Chrom. 890:3-13 (2000)) are preferred. For example, a Zorbax Poroshell 300SB-C3 microbore column (1.0×75 mm, 5 μm particles, Agilent Technologies, Inc., Palo Alto, Calif.) can be used. Columns packed with such superficially porous silica particles have excellent kinetic properties that allow for fast, stable, high resolution gradient chromatography of polypeptides, proteins, nucleic acids, DNA fragments etc. (Kirkland et al., Supra). The Poroshell particles can be manufactured from ultra-pure, less-acidic, Type B silica that is well suited for separating polar macromolecules, e.g., polypeptides and proteins. For other applications, e.g., vitamin analysis, other types of silica particles can be useful. For example, a HSS T3 column (2.1×150 mm, 1.8 μm particles, Waters Corp., Milford, Mass.) can be used. Any column chemistries with similar capabilities can be used. Any sub-2 μm particle, e.g. high strength silica core, with an attached C1-C18 ligand that is capable of separating highly polar compounds can be used for evaluating a vitamin process characteristic in accordance with the invention.

Various mobile phases can be used. The mobile phase used includes buffers without ion pairing agents, e.g., acetonitrile (e.g., from EM Science, Gibbstown, N.J. or from Burdick Jackson, Muskegon, Mich.) and water (e.g., HPLC grade, e.g., from EM Science). Ion paring agents include formate, acetate, and salts. Gradients of the buffers can be used, e.g., if two buffers are used, the concentration or percentage of the first buffer can decrease while the concentration or percentage of the second buffer increases over the course of the chromatography run. For example, the percentage of the first buffer can decrease from about 100%, about 99%, about 95%, about 90%, about 85%, about 80%, about 75%, about 70%, about 65%, about 60%, about 50%, about 45%, or about 40% to about 0%, about 1%, about 5%, about 10%, about 15%, about 20%, about 25%, about 30%, about 35%, or about 40% over the course of the chromatography run. As another example, the percentage of the second buffer can increase from about 0%, about 1%, about 5%, about 10%, about 15%, about 20%, about 25%, about 30%, about 35%, or about 40% to about 100%, about 99%, about 95%, about 90%, about 85%, about 80%, about 75%, about 70%, about 65%, about 60%, about 50%, about 45%, or about 40% over the course of the same run. Optionally, the concentration or percentage of the first and second buffer can return to their starting values at the end of the chromatography run. As an example, the percentage of the first buffer can change in five steps from 85% to 63% to 59% to 10% to 85%; while the percentage of the second buffer in the same steps changes from 15% to 37% to 41% to 90% to 15%. The percentages can change gradually as a linear gradient or in a non-linear (e.g., stepwise) fashion. For example, the gradient can be multiphasic, e.g., biphasic, triphasic, etc. For some applications, e.g., protein analysis, mobile phase A can be trifluoroacetic acid (TFA) (e.g., 0.5% v/v) in water and mobile phase B can be TFA (e.g., 0.5% v/v) in acetonitrile. For other applications, e.g., vitamin analysis, mobile phase A can be ammonium acetate (e.g., 15 mM, pH 5.8) in water and mobile phase B can be ammonium acetate (e.g., 15 mM, pH 5.8) in acetonitrile/water (e.g., 60% acetonitrile and 40% water).

The analyte, e.g., polypeptide and/or vitamin, can be analyzed by monitoring UV absorbance of the eluted fraction. For example, different UV wavelengths are used to maximize the sensitivity of individual vitamins.

The HPLC methods described herein are compatible with mass spectrometry (MS). For use with MS, buffers free of ion pairing agents can be employed, or the concentration of ion pairing agents (e.g., ammonium acetate) can be lowered in the mobile phases (e.g., to 5 mM). When buffers free of ion pairing agents are employed peaks isolated from HPLC can be directly analyzed without further purification (e.g., no desalting step is required). In addition, if no ion pairing agents are used, minor peaks from HPLC can be eluted, concentrated, and analyzed (e.g., off-line by MALDI) without further purification. Purification steps that are omitted include one or more of ion pairing agent removal (e.g., desalting), dialysis, drying.

The column temperature can be maintained at a constant temperature throughout the chromatography run, e.g., using a commercial column heater. In some embodiments, the column is maintained at a temperature between about 18° C. to about 70° C., e.g., about 30° C. to about 60° C., about 40° C. to about 50° C., e.g., at about 20° C., about 25° C., about 30° C., about 35° C., about 40° C., about 45° C., about 50° C., about 55° C., about 60° C., about 65° C., or about 70° C. For some applications, e.g., protein analysis, the preferred temperature is about 65° C. For some applications, e.g., vitamin analysis, the preferred temperature is about 45° C.

The flow rate of the mobile phase can be between about 0 to about 300 μL/min. For example the flow rate can be about 100 μL/min or about 300 μL/min. Substituting a column having the same packing and the same length, but a smaller diameter requires a reduction in the flow rate in order to retain the same retention time and resolution for peaks as seen with a column of wider diameter.

The run time for an HPLC method described herein can be between about 15 to about 240 minutes, e.g., about 20 to about 70 min, about 30 to about 60 min, about 40 to about 90 min, about 50 min to about 100 min, about 60 to about 120 min, about 50 to about 80 min. For some applications, e.g., protein analysis, the run time can be about 30 min. In some applications, e.g., vitamin analysis, the run time can be about 35 min.

The HPLC analyzed processes and products can further be subjected to analysis by mass spectrometry. Examples of mass spectrometry that can be used to further analyze a process characteristic include ESI-MS, turbospray ionization mass spectrometry, nanospray ionization mass spectrometry, thermospray ionization mass spectrometry, sonic spray ionization mass spectrometry, SELDI-MS and MALDI-MS. For example, the methods described herein can be used to provide LC-evaluated glycans for on-line mass spectrometry (e.g., ESI-MS) and/or for off-line mass spectrometry (e.g., MALDI-MS).

In addition, processes and preparation analyzed by the methods described herein can be subjected to further analysis by other analytical techniques such as weak cation exchange high performance liquid chromatography (wCEX-HPLC) and/or size exclusion high performance liquid chromatography (SE-HPLC).

### Electrospray Ionization-Quadrupole Time-of-Flight Mass Spectrometry (ESI-QTOF MS)

The methods described herein include coupling the HPLC method, e.g., the UPLC method, online with a mass spectrometer, e.g., a quadrupole mass spectrometer, a time of flight mass spectrometer or a quadrupole time-of-flight (QTOF) mass spectrometer with electrospray ionization (ESI), e.g., Q-Tof API US (Waters, Beverly, Mass.).

### Ionization Source

The effluent, or at least a portion of effluent, from the LC system can be directed to the ionization source of the spectrometer. The ionization used is preferably ESI. The source can be nanospray ionization such as a Z-spray ion source. ESI is one of the atmospheric pressure ionization (API) techniques and is well-suited to the analysis of polar molecules ranging from less than 100 Da to more than 1,000,000 Da in molecular mass. Nanospray ionization (M. Wilm and M. Mann, 1996, Anal. Chem., 68:1-8) is a slow flow rate version of electrospray ionization.

Generally, during standard ESI (M. Yamashita and J. Fenn, 1984, J. Phys. Chem. 88:4451-4459), the sample can be dissolved in a polar, volatile solvent (e.g., 0.05% trifluoroacetic acid (TFA) in water) to give a concentration of e.g., 0.20 μg/μL. Other solvents may be used, however, they should typically be of a high quality that is capable of being used without further purification. Samples, dissolved in a polar, volatile solvent, are pumped through, e.g., a narrow, stainless steel capillary (75-150 micrometers i.d.) at a flow rate of between, e.g., 1 μL/min and 1 mL/min e.g., 100 μL/min. A high voltage of, e.g., about 3 or 4 kV can be applied to the tip of the capillary, which is situated within the ionization source of the mass spectrometer, and as a consequence of this strong electric field, the sample emerging from the tip can be dispersed into an aerosol of highly charged droplets, a process that can be aided by a co-axially introduced nebulizing gas flowing around the outside of the capillary. This gas, e.g., nitrogen, helps to direct the spray emerging from the capillary tip towards the mass spectrometer. The charged droplets can diminish in size by solvent evaporation, assisted by a warm flow of the drying gas, e.g., nitrogen, which passes across the front of the ionization source. Eventually, charged sample ions, free from solvent, can be released from the droplets, some of which can pass through a sampling cone or orifice into an intermediate vacuum region, and from there through a small aperture into the analyzer of the mass spectrometer, which can be held under high vacuum. The lens voltages can be optimized individually for each sample.

In a positive ionization mode, a trace of formic acid can be added to aid protonation of the sample molecules; in a negative ionization mode a trace of ammonia solution or a volatile amine can be added to aid deprotonation of the sample molecules. The preferred mode of the present methods is a positive ionization mode.

### The Analyzer

The main function of the mass analyzer is to separate, or resolve, the ions formed in the ionization source of the mass spectrometer according to their mass-to-charge (m/z) ratios. There are a number of mass analyzers currently available, e.g., quadrupoles, time-of-flight (TOF) analyzers, magnetic sectors, and both Fourier transform and quadrupole ion traps. The TOF analyzer uses an electric field to accelerate the ions through the same potential, and then measures the time they take to reach the detector. If the particles all have the same charge, then their kinetic energies will be identical, and their velocities will depend only on their masses. Lighter ions will reach the detector first. Quadrupole mass analyzers use oscillating electrical fields to selectively stabilize or destabilize ions passing through a radio frequency (RF) quadrupole field. A quadrupole mass analyzer acts as a mass selective filter and is closely related to the quadrupole ion trap, particularly the linear quadrupole ion trap except that it operates without trapping the ions. A common variation of the quadrupole is the triple quadrupole.

The preferred spectrometer of the present methods is a tandem (MS-MS) spectrometer that includes both a quadrupole and a TOF analyzer, or a QTOF analyzer. The two analyzers can be separated by a collision cell into which an inert gas, e.g., argon or xenon, is admitted to collide with the selected sample ions and to bring about their fragmentation. The collision energy can be, e.g., 5 eV and 10 eV. The desolvation and ion source block temperatures can be, e.g., about 275° C. and 115° C., respectively. The ESI capillary voltage can be, e.g., about 3000 V, and the pusher cycle time can be, e.g., 88 μseconds.

### The Detector

The detector of the spectrometer monitors the ion current, amplifies it and transmits the signal to the data system, where it is recorded in the form of mass spectra. The m/z values of the ions are plotted against their intensities to show the number of components in the sample, the molecular mass of each component, and the relative abundance of the various components in the sample. The type of detector is supplied to suit the type of analyzer and can include the photomultiplier, the electron multiplier and the micro-channel plate detectors. In the present methods, the data can be acquired, e.g., from m/z 50 to m/z 3000; e.g., from m/z 500 to m/z 4300, in, e.g., 2 second scans, with, e.g., 0.1 second interscan delay.

Instrument control, data acquisition, calibration, and mass spectrum processing can be performed with e.g., MassLynx 3.5 software (Waters, Beverly, Mass.). Multiply-charged mass data can be deconvoluted into a zero-charge mass spectrum using probabilistic maximum entropy analysis with e.g., MaxEnt module (MicroMass MS Technologies, Waters Corp., Milford, Mass.). Theoretical mass values for intact proteins and subunits based on the predicted amino acid sequences and PTMs (post translational modifications) can be determined with Protein analysis worksheet (PAWS), version 2000.06.08 (Genomic Solutions, Ann Arbor, Mich.).

### MALDI-MS

Methods described herein can include the use of MALDI-MS to analyze process characteristics from the LC effluent. In one embodiment, the MALDI ion source uses a time of flight (TOF) mass analyzer, a quadrupole mass analyzer or a quadrupole time of flight (Q-TOF). The MALDI-TOF can be linear time of flight (L-TOF) (e.g., with continuous or delayed ion extraction) or reflection time of flight (re-TOF). Preferably, L-TOF is used with a delayed ion extraction.

Prior to MALDI-MS analysis, the HPLC effluent, e.g., fractions obtained by, separated by or purified by HPLC, is combined with a matrix. The matrix can include one or more components. For example, the matrix can include an inorganic compound, e.g., an inorganic compound having high molar absorptivity at the wavelength of the laser being used. Examples of matrices that can be used include 5-dihydroxybenzoic acid (DHB) and 2-hydroxy-5-methoxybenzoic acid (HMB).

The preparation, and/or the matrix can be deposited on the MALDI sample plate using known methods such as dried droplet method, surface preparation methods, crushed crystal methods and electrospray deposition. In some embodiments, the preparation and/or matrix is contacted with a solvent, H₂O or a combination thereof prior to being deposited on the sample plate. Examples of solvents include acetonitrile.

Separation of ions of different m/z values in TOF mass spectrometry can be measured by the total time of flight from ion formation to impact on a detector.

### Polypeptides

Polypeptides in the preparations can be produced recombinantly. The terms “recombinantly expressed protein or polypeptide” and “recombinant protein or polypeptide” as used herein refer to a polypeptide expressed from a host cell that has been manipulated by the hand of man to express that polypeptide. In certain embodiments, the host cell is a mammalian cell. In certain embodiments, this manipulation may comprise one or more genetic modifications. For example, the host cells may be genetically modified by the introduction of one or more heterologous genes encoding the polypeptide to be expressed. The heterologous recombinantly expressed polypeptide can be identical or similar to polypeptides that are normally expressed in the host cell. The heterologous recombinantly expressed polypeptide can also be foreign to the host cell, e.g., heterologous to polypeptides normally expressed in the host cell. In certain embodiments, the heterologous recombinantly expressed polypeptide is chimeric. For example, portions of a polypeptide may contain amino acid sequences that are identical or similar to polypeptides normally expressed in the host cell, while other portions contain amino acid sequences that are foreign to the host cell. Additionally or alternatively, a polypeptide may contain amino acid sequences from two or more different polypeptides that are both normally expressed in the host cell. Furthermore, a polypeptide may contain amino acid sequences from two or more polypeptides that are both foreign to the host cell. In some embodiments, the host cell is genetically modified by the activation or upregulation of one or more endogenous genes.

Any protein that is recombinantly produced can be used in accordance with the present invention. For example, the methods described herein may be employed to analyze any pharmaceutically or commercially relevant antibody, receptor, cytokine, growth factor, enzyme, clotting factor, hormone, regulatory factor, antigen, binding agent, among others, and methods of making such polypeptides. The following list of proteins that can be analyzed according to the present invention is merely exemplary in nature, and is not intended to be a limiting recitation. One of ordinary skill in the art will understand that any polypeptide produced in cell culture or a process bioreactor can be evaluated and will be able to select the particular polypeptide to be produced as needed.

### Antibodies and Binding Fragments

The methods disclosed herein can be used to evaluate post translational structures in a preparation of antibody or an antigen-binding fragment thereof. Antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG₁, IgG₂, IgG₃, IgG₄, IgA₁, and IgA₂. Each light chain includes an N-terminal variable (V) domain (VL) and a constant (C) domain (CL). Each heavy chain includes an N-terminal V domain (VH), three or four C domains (CHs), and a hinge region. The CH domain most proximal to VH is designated as CH1. Often, the VH domain of an antibody is glycosylated, e.g., with an N-linked glycan. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is typically the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit the binds to the antigen, i.e., the antigen-binding fragment, or, e.g., the portion of the CH subunit that binds to and/or activates, e.g., an Fc receptor and/or complement. The CDRs typically refer to the Kabat CDRs, as described in Sequences of Proteins of Immunological Interest, US Department of Health and Human Services, 1991, eds. Kabat et al. Another standard for characterizing the antigen binding site is to refer to the hypervariable loops as described by Chothia. See, e.g., Chothia, D. et al., 1992, J. Mol. Biol. 227:799-817, and Tomlinson et al., 1995, EMBO J. 14:4628-4638. Still another standard is the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Embodiments described with respect to Kabat CDRs can alternatively be implemented using similar described relationships with respect to Chothia hypervariable loops or to the AbM-defined loops. As used herein, the term “antibody” includes a protein comprising at least one, and typically two, VH domains or portions thereof, and/or at least one, and typically two, VL domains or portions thereof. In certain embodiments, the antibody is a tetramer of two heavy immunoglobulin chains and two light immunoglobulin chains, wherein the heavy and light immunoglobulin chains are inter-connected by, e.g., disulfide bonds. The antibodies, or a portion thereof, can be obtained from any origin, including, but not limited to, rodent, primate (e.g., human and non-human primate), camelid, as well as recombinantly produced, e.g., chimeric, humanized, and/or in vitro generated, as described in more detail herein.

Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) a camelid or camelized variable domain, e.g., a VHH domain; (vii) a single chain Fv (scFv); (viii) a bispecific antibody; and (ix) one or more antigen binding fragments of an immunoglobulin fused to an Fc region. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. Science 242:423-426 (1988); Huston et al. Proc. Natl. Acad. Sci. U.S.A. 85:5879-5883 (1988)). Such single chain antibodies are also intended to be encompassed within the term “antigen-binding fragment” of an antibody. These antibody fragments are obtained using conventional techniques known to those skilled in the art, and the fragments are evaluated for function in the same manner as are intact antibodies.

Small Modular ImmunoPharmaceuticals (SMIP™) provide an example of a variant molecule comprising a binding domain polypeptide. SMIPs and their uses and applications are disclosed in, e.g., U.S. Published Patent Application Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine and shark. According to one aspect of the invention, a single domain antibody as used herein is a naturally occurring single domain antibody known as heavy chain antibody devoid of light chains. Such single domain antibodies are disclosed in PCT publication No. WO 9404678 for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., S. Songsivilai and P. J. Lachmann, 1990, Clin. Exp. Immunol. 79:315-321, and Kostelny et al., 1992, J. Immunol. 148, 1547-1553.

Numerous methods known to those skilled in the art are available for obtaining antibodies. For example, monoclonal antibodies may be produced by generation of hybridomas in accordance with known methods. Hybridomas formed in this manner are then screened using standard methods, such as enzyme-linked immunosorbent assay (ELISA) and surface plasmon resonance (Biacore™) analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

One exemplary method of making antibodies includes screening protein expression libraries, e.g., phage or ribosome display libraries. Phage display is described, for example, in Ladner et al., U.S. Pat. No. 5,223,409; G. P. Smith, 1985, Science 228:1315-1317; and PCT Publication Nos. WO 92/18619; WO 91/17271; WO 92/20791; WO 92/15679; WO 93/01288; WO 92/01047; WO 92/09690; and WO 90/02809.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al., 1994, Nat. Genet. 7:13-21, U.S. Patent Publication No. 2003/0070185, PCT Publication No. WO 96/34096, published Oct. 31, 1996; and PCT Application No. PCT/US96/05928, filed Apr. 29, 1996.

In another embodiment, a monoclonal antibody that is obtained from the non-human animal, and then modified, e.g., humanized, deimmunized, chimeric, may be produced using recombinant DNA techniques known in the art. A variety of approaches for making chimeric antibodies have been described. See e.g., S. L. Morrison et al., 1984, Proc. Natl. Acad. Sci. U.S.A. 81:6851-6855; S. Takeda et al., 1985, Nature 314:452-454, Cabilly et al. U.S. Pat. No. 4,816,567; Boss et al. U.S. Pat. No. 4,816,397; Tanaguchi et al., European Patent Publication No. EP171496; European Patent Publication No. 0173494, United Kingdom Patent GB 2177096B. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies can be generated by replacing sequences of the Fv variable domain that are not directly involved in antigen binding with equivalent sequences from human Fv variable domains. Exemplary methods for generating humanized antibodies or fragments thereof are provided by S. L. Morrison, 1985, Science 229:1202-1207; by Oi et al., 1986, BioTechniques 4:214-221; and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or back mutations. Such altered immunoglobulin molecules can be made by any of several techniques known in the art, (e.g., Teng et al., 1983, Proc. Natl. Acad. Sci. U.S.A. 80:7308-7312; Kozbor et al., 1983, Immunol. Today 4: 7279; Olsson et al., 1982, Meth. Enzymol. 92: 3-16), and may be made according to the teachings of PCT Publication No. WO 92/06193 or European Patent Publication No. EP 0239400).

An antibody may also be modified by specific deletion of human T cell epitopes or “deimmunization” by the methods disclosed in PCT Publication No. WO 98/52976 and PCT Publication No. WO 00/34317. Briefly, the heavy and light chain variable domains of an antibody can be analyzed for peptides that bind to MHC Class II; these peptides represent potential T-cell epitopes (as defined in PCT Publication No. WO 98/52976 and PCT Publication No. WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in PCT Publication No. WO 98/52976 and PCT Publication No. WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences, e.g., are disclosed in Tomlinson, et al., 1992, J. Mol. Biol. 227:776-798; Cook, G. P. et al., 1995, Immunol. Today 16(5): 237-242; Chothia, D. et al., 1992, J. Mol. Biol. 227:799-817; and Tomlinson et al., 1995, EMBO J. 14:4628-4638. The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

In certain embodiments, an antibody can contain an altered immunoglobulin constant or Fc region. For example, an antibody produced in accordance with the teachings herein may bind more strongly or with more specificity to effector molecules such as complement and/or Fc receptors, which can control several immune functions of the antibody such as effector cell activity, lysis, complement-mediated activity, antibody clearance, and antibody half-life. Typical Fc receptors that bind to an Fc region of an antibody (e.g., an IgG antibody) include, but are not limited to, receptors of the FcγRI, FcγRII, and FcγRIII and FcRn subclasses, including allelic variants and alternatively spliced forms of these receptors. Fc receptors are reviewed in Ravetch and Kinet, 1991, Annu. Rev. Immunol. 9:457-492; Capel et al., 1994, Immunomethods 4:25-34; and de Haas et al., 1995 J. Lab. Clin. Med. 126:330-341.

### Receptors

Another class of polypeptides that can be evaluated in accordance with teachings of the present invention includes receptors. Given the biological importance of receptors and their importance as potential therapeutic agents, analysis of these molecules in accordance with the present invention is of particular interest.

Receptors are typically trans-membrane glycoproteins that function by recognizing an extra-cellular signaling ligand. Receptors often have a protein kinase domain in addition to the ligand recognizing domain. This protein kinase domain initiates a signaling pathway by phosphorylating target intracellular molecules upon binding the ligand, leading to developmental or metabolic changes within the cell. In certain embodiments, an extracellular domain of a transmembrane receptor is evaluated in accordance with methods and systems disclosed herein. In certain embodiments, an intracellular domain of a transmembrane receptor is evaluated in accordance with methods and systems disclosed herein.

In certain embodiments, tumor necrosis factor inhibitors, in the form of tumor necrosis factor alpha and beta receptors (TNFR-1; EP 417,563 published Mar. 20, 1991; and TNFR-2, EP 417,014 published Mar. 20, 1991, each of which is incorporated herein by reference in its entirety) are evaluated in accordance with systems and methods of the present invention (for review, see Naismith and Sprang, 1995-1996, J. Inflamm. 47(1-2):1-7, incorporated herein by reference in its entirety). According to some embodiments, a tumor necrosis factor inhibitor comprises a soluble TNF receptor. In certain embodiments, a tumor necrosis factor inhibitor comprises a soluble TNFR fused to any portion of an immunoglobulin protein, including the Fc region of an immunoglobulin. In certain embodiments, TNF inhibitors of the present invention are soluble forms of TNFR I and TNFR II. In certain embodiments, TNF inhibitors of the present invention are soluble TNF binding proteins. In certain embodiments, the TNF inhibitors of the present invention are TNFR-Fc, for example, etanercept. As used herein, “etanercept,” refers to a TNFR-Fc, which is a dimer of two molecules of the extracellular portion of the p75 TNF-α receptor, each molecule consisting of a 235 amino acid Fc portion of human IgG1.

In some embodiments, receptors to be evaluated in accordance with the present invention are receptor tyrosine kinases (RTKs). The RTK family includes receptors that are crucial for a variety of functions numerous cell types (see, e.g., Yarden and Ullrich, 1988, Ann. Rev. Biochem. 57:433-478; Ullrich and Schlessinger, 1990, Cell 61:243-254, each of which is incorporated herein by reference). Non-limiting examples of RTKs include tumor necrosis factor alpha and beta receptors, members of the fibroblast growth factor (FGF) receptor family, members of the epidermal growth factor receptor (EGF) family, platelet derived growth factor (PDGF) receptor, tyrosine kinase with immunoglobulin and EGF homology domains-1 (TIE-1) and TIE-2 receptors (Sato et al., 1995, Nature 376(6535):70-74, incorporated herein by reference in its entirety) and c-Met receptor, some of which have been suggested to promote angiogenesis, directly or indirectly (Mustonen and Alitalo, 1995, J. Cell Biol. 129:895-898). Other non-limiting examples of RTK's include fetal liver kinase 1 (FLK-1) (sometimes referred to as kinase insert domain-containing receptor (KDR) (Terman et al., 1991, Oncogene 6:1677-1683) or vascular endothelial cell growth factor receptor 2, VEGFR-2), fms-like tyrosine kinase-1 (Flt-1) (DeVries et al., 1992, Science 255:989-991; Shibuya et al., 1990, Oncogene 5:519-524), sometimes referred to as vascular endothelial cell growth factor receptor 1 (VEGFR-1), neuropilin-1, endoglin, endosialin, and Axl. Those of ordinary skill in the art will be aware of other receptors that can be evaluated in accordance with the present invention.

In certain embodiments, the receptor to be analyzed in accordance with the present invention is a G-protein coupled receptor (GPCR). GPCRs are a major target for drug action and development. In fact, receptors have led to more than half of the currently known drugs (Drews, 1996, Nat. Biotechnol. 14:1516-1518) and GPCRs represent the most important target for therapeutic intervention with 30% of clinically prescribed drugs either antagonizing or agonizing a GPCR (Milligan, G. and Rees, S., 1999, Trends Pharmacol. Sci. 20:118-124). Since these receptors have an established, proven history as therapeutic targets, analysis of GPCRs in accordance with the present invention is also of particular interest.

### Fusions Proteins

Fusion proteins generally have all or a substantial portion of a targeting peptide, linked at the N- or C-terminus, to all or a portion of a second polypeptide or protein. For example, fusions may employ leader sequences from other species to permit the recombinant expression of a protein in a heterologous host. Another useful fusion includes the addition of an immunologically active domain, such as an antibody epitope, to facilitate purification of the fusion protein.

The fusion protein can include cytostatic proteins, cytocidal proteins, pro-apoptosis agents, anti-angiogenic agents, hormones, cytokines, growth factors, peptide drugs, antibodies, Fab fragments antibodies, antigens, receptor proteins, enzymes, lectins, MHC proteins, cell adhesion proteins and binding proteins. In certain embodiments, the fusion protein can include a targeting moiety, e.g., a soluble receptor fragment or a ligand, and an immunoglobulin chain, an Fc fragment, a heavy chain constant regions of the various isotypes, including: IgG1, IgG2, IgG3, IgG4, IgM, IgA1, IgA2, IgD, and IgE). For example, the fusion protein can include the extracellular domain of a receptor, and, e.g., fused to, a human immunoglobulin Fc chain (e.g., human IgG, e.g., human IgG1 or human IgG4, or a mutated form thereof). In one embodiment, the human Fc sequence has been mutated at one or more amino acids, e.g., mutated at residues 254 and 257 from the wild type sequence to reduce Fc receptor binding. The fusion proteins may additionally include a linker sequence joining the first moiety to the second moiety, e.g., the immunoglobulin fragment. For example, the fusion protein can include a peptide linker, e.g., a peptide linker of about 4 to 20, more preferably, 5 to 10, amino acids in length; the peptide linker is 8 amino acids in length. For example, the fusion protein can include a peptide linker having the formula (Ser-Gly-Gly-Gly-Gly)y wherein y is 1, 2, 3, 4, 5, 6, 7, or 8. In other embodiments, additional amino acid sequences can be added to the N- or C-terminus of the fusion protein to facilitate expression, steric flexibility, detection and/or isolation or purification.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.), 1992, Current Protocols in Molecular Biology, John Wiley & Sons). Moreover, many expression vectors are commercially available that encode a fusion moiety (e.g., an Fc region of an immunoglobulin heavy chain). Immunoglobulin fusion polypeptides are known in the art and are described in e.g., U.S. Pat. Nos. 5,516,964; 5,225,538; 5,428,130; 5,514,582; 5,714,147; and 5,455,165.

### Growth Factors and Cytokines

Another class of polypeptides that can be analyzed for process characteristics includes growth factors and other signaling molecules, such as cytokines.

Growth factors are typically glycoproteins that are secreted by cells and bind to and activate receptors on other cells, initiating a metabolic or developmental change in the receptor cell. Non-limiting examples of mammalian growth factors and other signaling molecules include cytokines; epidermal growth factor (EGF); platelet-derived growth factor (PDGF); fibroblast growth factors (FGFs) such as aFGF and bFGF; transforming growth factors (TGFs) such as TGF-alpha and TGF-beta, including TGF-beta 1, TGF-beta 2, TGF-beta 3, TGF-beta 4, or TGF-beta 5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (TLs), e.g., IL-1 to IL-13 (e.g., IL-11); tumor necrosis factor (TNF) alpha and beta; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin, hemopoietic growth factor; enkephalinase; RANTES (regulated on activation normally T-cell expressed and secreted); human macrophage inflammatory protein (MIP-1-alpha); mullerian-inhibiting substance; relaxin A-chain; relaxin B-chain; prorelaxin; mouse gonadotropin-associated peptide; neurotrophic factors such as bone-derived neurotrophic factor (BDNF), neurotrophin-3, -4, -5, or -6 (NT-3, NT-4, NT-5, or NT-6), or a nerve growth factor such as NGF-beta. One of ordinary skill in the art will be aware of other growth factors or signaling molecules that can be expressed in accordance with methods and compositions of the present invention.

Specific alterations in the post translational structures (e.g., glycan structure) of growth factors or other signaling molecules have been shown to have dramatic effects on their therapeutic properties. As one example, a common method of treatment for patients who suffer from chronic anemia is to provide them with frequent injections of recombinant human erythropoietin (rHuEPO) in order to boost their production of red blood cells. An analog of rHuEPO, darbepoetin alfa (Aranesp®), has been developed to have a longer duration than normal rHuEPO. The primary difference between darbepoetin alfa and rHuEPO is the presence of two extra sialic-acid-containing N-linked oligosaccharide chains. Production of darbepoetin alfa has been accomplished using in vitro glycoengineering (see Elliott et al., Nat. Biotechnol. 21(4):414-421, 2003, incorporated herein by reference in its entirety). Elliott et al. used in vitro mutagenesis to incorporate extra glycosylation sites into the rHuEPO polypeptide backbone, resulting in expression of the darbepoetin alfa analog. The extra oligosaccharide chains are located distal to the EPO receptor binding site and apparently do not interfere with receptor binding. However, darbepoetin alfa's half-life is up to three-fold higher than rHuEPO, resulting in a much more effective therapeutic agent. Thus, methods of determining post translational structures such as glycan structures, e.g., between polypeptides such as cytokines and growth factors produced, e.g., by alternative processes or expression systems, can be useful to evaluate potential differences in activity.

### Clotting Factors

Clotting factors can also be evaluated for process characteristics associated with the production of the clotting factors. Hemophilia B is a disorder in which the blood of the sufferer is unable to clot. Thus, any small wound that results in bleeding is potentially a life-threatening event. For example, Coagulation Factor IX (Factor IX or “FIX”) is a single-chain glycoprotein whose deficiency results in Hemophilia B. FIX is synthesized as a single chain zymogen that can be activated to a two-chain serine protease (Factor IXa) by release of an activation peptide. The catalytic domain of Factor IXa is located in the heavy chain (see Chang et al., 1997, J. Clin. Invest. 100(4):886-892, incorporated herein by reference in its entirety). FIX has multiple glycosylation sites including both N-linked and O-linked carbohydrates. One particular O-linked structure at Serine 61 (Sia-α2,3-Gal-β1,4-GlcNAc-β1,3-Fuc-α1-O-Ser) was once thought unique to FIX but has since found on a few other molecules including the Notch protein in mammals and Drosophila (Moloney et al., 2000, J. Biol. Chem. 275(13):9604-9611). FIX produced by Chinese Hamster Ovary (“CHO”) cells in cell culture exhibits some variability in the Serine 61 oligosaccharide chain. These different glycoforms, and other potential glycoforms, may have different abilities to induce clotting when administered to humans or animals and/or may have different stabilities in the blood, resulting in less effective clotting.

Hemophilia A, which is clinically indistinguishable from Hemophilia B, is caused by a defect in human clotting factor VIII, another glycoprotein that is synthesized as a single chain and then processed into a two-chain active form. The present invention may used to evaluate post translational structures associated with various preparations to determine, e.g., effect of the structures in the preparation on clotting activity. Other clotting factors that can be analyzed by the methods described herein include tissue factor and von Willebrands factor.

### Enzymes

Another class of polypeptides that can be analyzed for process characteristics according to the invention includes enzymes. Enzymes may be glycoproteins whose glycosylation pattern affects enzymatic activity. Thus, the present invention may also be used to analyze enzymes produced in a cell culture, e.g., under different cell culture conditions and/or expression systems, to provide enzymes that have a more extensive or otherwise more desirable post-translational properties.

For example, a deficiency in glucocerebrosidase (GCR) results in a condition known as Gaucher's disease, which is caused by an accumulation of glucocerebrosidase in lysosomes of certain cells. Subjects with Gaucher's disease exhibit a range of symptoms including splenomegaly, hepatomegaly, skeletal disorder, thrombocytopenia and anemia. Friedman and Hayes showed that recombinant GCR (rGCR) containing a single substitution in the primary amino acid sequence exhibited an altered glycosylation pattern, specifically an increase in fucose and N-acetyl glucosamine residues compared to naturally occurring GCR (see U.S. Pat. No. 5,549,892).

Friedman and Hayes also demonstrated that this rGCR exhibited improved pharmacokinetic properties compared to naturally occurring rGCR. For example, approximately twice as much rGCR targeted liver Kupffer cells than did naturally occurring GCR. Although the primary amino acid sequences of the two proteins differed at a single residue, Friedman and Hayes hypothesized that the altered glycosylation pattern of rGCR may also influence the targeting to Kupffer cells. One of ordinary skill in the art will be aware of other known examples of enzymes that exhibit altered enzymatic, pharmacokinetic and/or pharmacodynamic properties resulting from an alteration in their post translational properties.

### Production Parameters:

Methods described herein include evaluating a production parameter or parameters and, optionally selecting a production parameter or parameters such that a desired polypeptide preparation is produced. By using information regarding the effects of various process characteristics on the polypeptide preparation, production parameters can be selected prior to the production of a polypeptide preparation that correlate with the desired polypeptide preparation properties. A production parameter as used herein is a parameter or element in a production process. Production parameters that can be selected include, e.g., the cell or cell line used to produce the polypeptide preparation, the vector or vector elements used to produce the polypeptide preparation, the culture medium, culture process or bioreactor variables (e.g., batch, fed-batch, or perfusion), purification process and formulation of a polypeptide preparation.

Production parameters include: 1) the types of host; 2) genetics of the host; 3) media type; 4) fermentation platform; 5) purification steps; and 6) formulation. Other production parameters include: selection of host subclones based on desired polypeptide preparation properties; regulation of host gene levels constitutive or inducible; introduction of novel genes or promoter elements; media components, e.g. media components described herein; physiochemical growth properties such as those described herein; growth vessel type (e.g. bioreactor type, T flask); cell density; cell cycle; enrichment of product with a desired isotype (e.g. by lectin or antibody-mediated enrichment, ion-exchange chromatography, capillary electrophoresis (CE), or similar method); or other production parameters clear to someone skilled in the art.

### Polypeptide Production

The invention includes methods of evaluating a polypeptide preparation by comparing a value for a process characteristic to a preselected criterion, e.g., a preselected value or range for the process characteristic for the same or different production protocols, e.g. the same or different production parameter or parameters. Production of the polypeptide preparations can vary, e.g., based on culture conditions (e.g., temperature, media, plating techniques), isolation techniques, and expression systems (e.g., recombinant versus native expression, or recombinant expression from different types of host cells, e.g., CHO versus COS cell expression). Such variation can result in the absence, presence or changes in the amount of a polypeptide or an isoform of the polypeptide (e.g., major, minor and trace isoforms of the polypeptide), as well as other process related biomolecules (e.g., vitamins and other media components).

Recombinant methods of producing polypeptides are known in the art. Nucleotide sequences encoding the proteins are typically inserted in an expression vector for introduction into host cells that may be used to produce the desired quantity of polypeptides. The term “vector” includes a nucleic acid construct often including a nucleic acid, e.g., a gene, and further including minimal elements necessary for nucleic acid replication, transcription, stability and/or protein expression or secretion from a host cell. Such constructs may exist as extrachromosomal elements or may be integrated into the genome of a host cell.

The term “expression vector” includes a specific type of vector wherein the nucleic acid construct is optimized for the high-level expression of a desired protein product. Expression vectors often have transcriptional regulatory agents, such as promoter and enhancer elements, optimized for high-levels of transcription in specific cell types and/or optimized such that expression is constitutive based upon the use of a specific inducing agent. Expression vectors further have sequences that provide for proper and/or enhanced translation of the protein. As known to those skilled in the art, such vectors may easily be selected from the group consisting of plasmids, phages, viruses, and retroviruses. The term “expression cassette” includes a nucleic acid construct containing a gene and having elements in addition to the gene that allow for proper and or enhanced expression of that gene in a host cell. For producing antibodies, nucleic acids encoding light and heavy chains can be inserted into expression vectors. Such sequences can be present in the same nucleic acid molecule (e.g., the same expression vector) or alternatively, can be expressed from separate nucleic acid molecules (e.g., separate expression vectors).

The term “operably linked” includes a juxtaposition wherein the components are in a relationship permitting them to function in their intended manner (e.g., functionally linked). As an example, a promoter/enhancer operably linked to a polynucleotide of interest is ligated to said polynucleotide such that expression of the polynucleotide of interest is achieved under conditions which activate expression directed by the promoter/enhancer.

Expression vectors are typically replicable in the host organisms either as episomes or as an integral part of the host chromosomal DNA. Commonly, expression vectors contain selection markers (e.g., ampicillin-resistance, hygromycin-resistance, tetracycline resistance, kanamycin resistance or neomycin resistance) to permit detection of those cells transformed with the desired DNA sequences (see, e.g., Itakura et al., U.S. Pat. No. 4,704,362). In addition to the immunoglobulin DNA cassette sequences, insert sequences, and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin, or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

Once the vector has been incorporated into the appropriate host cell, the host cell is maintained under conditions suitable for high level expression of the nucleotide sequences, and the collection and purification of the desired antibodies. Various host cells can be utilized to produce a polypeptide, and using methods disclosed herein the various isoforms and process related biomolecules associated with expression in different host cells can be evaluated. In certain embodiments, the host cell is mammalian. Non-limiting examples of mammalian cells that may be used in accordance with the present invention include BALB/c mouse myeloma line (NSO/I, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., 1977, J. Gen. Virol. 36:59); baby hamster kidney cells (BHK, ATCC CCL 10); Chinese hamster ovary cells +/−DHFR (CHO, Urlaub and Chasin, 1980, Proc. Natl. Acad. Sci. U.S.A. 77:4216); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68, 1982); MRC 5 cells; FS4 cells; and a human hepatoma line (Hep G2).

Additionally, any number of commercially and non-commercially available hybridoma cell lines that express polypeptides or proteins may be used to produce a polypeptide preparation. One skilled in the art will appreciate that hybridoma cell lines might have different nutrition requirements and/or might require different culture conditions for optimal growth and polypeptide or protein expression, and will be able to modify conditions as needed. In addition, using the methods disclosed herein, the effect, if any, of varying culture conditions can be evaluated to determine if the conditions produce varying process characteristics and/or varying levels of process characteristics.

Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. (See, e.g., Co et al., (1992) J. Immunol. 148:1149). Preferred regulatory sequences for mammalian host cell expression include viral elements that direct high levels of protein expression in mammalian cells, such as promoters and/or enhancers derived from FF-1a promoter and BGH poly A, cytomegalovirus (CMV) (such as the CMV promoter/enhancer), Simian Virus 40 (SV40) (such as the SV40 promoter/enhancer), adenovirus (e.g., the adenovirus major late promoter (AdMLP)), and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al. In exemplary embodiments, the antibody heavy and light chain genes are operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. In exemplary embodiments, the construct include an internal ribosome entry site (IRES) to provide relatively high levels of polypeptides of the invention in eukaryotic host cells. Compatible IRES sequences are disclosed in U.S. Pat. No. 6,193,980 that is also incorporated herein.

Alternatively, coding sequences can be incorporated in a transgene for introduction into the genome of a transgenic animal and subsequent expression in the milk of the transgenic animal (see, e.g., Deboer et al., U.S. Pat. No. 5,741,957; Rosen, U.S. Pat. No. 5,304,489; and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

Prokaryotic host cells may also be suitable for producing the antibodies of the invention. E. coli is one prokaryotic host particularly useful for cloning the polynucleotides (e.g., DNA sequences) of the present invention. Other microbial hosts suitable for use include bacilli, such as Bacillus subtilis, enterobacteriaceae, such as Escherichia, Salmonella, and Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Expression of proteins in prokaryotes is most often carried out in E. coli with vectors containing constitutive or inducible promoters directing the expression of either fusion or non-fusion proteins. Fusion vectors add a number of amino acids to an antibody encoded therein, often to the constant region of the recombinant antibody, without affecting specificity or antigen recognition of the antibody. Addition of the amino acids of the fusion peptide can add additional function to the antibody, for example as a marker (e.g., epitope tag such as myc or flag).

Other microbes, such as yeast, are also useful for expression. Saccharomyces is a preferred yeast host, with suitable vectors having expression control sequences (e.g., promoters), an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

A cell can be selected for production of a polypeptide based, e.g., upon attributes of the cell itself which produce or show a preference for production of the desired process characteristic or characteristics. Attributes of the cell that may effect post translational structures include the type of cell, cell state, the cell cycle, the passage number, and the metabolic stress level of the cell.

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally, Sambrook et al., Molecular Cloning: A Laboratory Manual (Cold Spring Harbor Press, 2nd ed., 1989), incorporated by reference herein in its entirety for all purposes.). Other methods used to transform mammalian cells include the use of polybrene, protoplast fusion, liposomes, electroporation, and microinjection (see generally, Sambrook et al., supra).

When heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms of the present invention can be separated as described herein and/or further purified according to procedures known in the art, including ammonium sulfate precipitation, affinity columns, column chromatography, HPLC purification, gel electrophoresis and the like (see generally Scopes, Protein Purification (Springer-Verlag, N.Y., (1982)). Substantially pure immunoglobulins of at least about 90 to 95% homogeneity are preferred, and 98 to 99% or more homogeneity most preferred, for pharmaceutical uses.

### Culture Media and Processing

The methods described herein can include determining and/or selecting media components or culture conditions which result in the production of a desired process characteristic, e.g., the production of a desired polypeptide and/or polypeptide isoform, a desired profile of a polypeptide and/or polypeptide isoform including the amount of production of a desired polypeptide and/or polypeptide isoform or a desired profile of a polypeptide and/or polypeptide isoform. Culture parameters that can be determined include media components, pH, feeding conditions, osmolarity, carbon dioxide levels, agitation rate, temperature, cell density, seeding density, timing and sparge rate.

Changes in production parameters such as the speed of agitation of a cell culture, the temperature at which cells are cultured, the components in the culture medium, the times at which cultures are started and stopped, or variation in the timing of nutrient supply can result in variation of a process characteristic of the produced polypeptide product. Thus, methods described herein can include one or more of: increasing, decreasing or maintaining the speed at which cells are agitated, increasing, decreasing or maintaining the temperature at which cells are cultured, adding, removing or maintaining media components, and altering or maintaining the times at which cultures are started and/or stopped.

### Media

The methods described herein can include determining and/or selecting a media component and/or the concentration of a media component that has a positive correlation to a desired process characteristic, e.g., the desired polypeptide and/or polypeptide isoform or the desired profile of polypeptide and/or polypeptide isoforms. A media component can be added in or administered over the course of polypeptide production or when there is a change in media, depending on culture conditions. Media components include components added directly to culture as well as components that are a byproduct of cell culture.

Media components include, e.g., buffer, amino acid content, vitamin content, salt content, mineral content, serum content, carbon source content, lipid content, nucleic acid content, hormone content, trace element content, ammonia content, co-factor content, indicator content, small molecule content, hydrolysate content and enzyme modulator content. Examples of various media components that can be selected include vitamins, carbon source (natural or unnatural, salts, sugars, sera, sodium pyruvate, surfactants, ammonia, lipids, hormones or growth factors, buffers, amino acids, sugar precursors, indicators, nucleosides or nucleotides, butyrate, animal derived products, gene inducers, regulators of pH, betaine or osmoprotectant, trace elements and minerals.

Exemplary buffers include Tris, Tricine, HEPES, MOPS, PIPES, TAPS, bicine, BES, TES, cacodylate, MES, acetate, MKP, ADA, ACES, glycinamide and acetamidoglycine.

The media can be serum free (e.g., in embodiments were the polypeptide is analyzed or vitamin content is analyzed), or can include animal derived products (e.g., in embodiments where the vitamin content is analyzed). Examples of animal derived products include fetal bovine serum (FBS), fetal calf serum (FCS), horse serum (HS), human serum, animal derived serum substitutes (e.g., Ultroser G, SF and HY; non-fat dry milk; Bovine EX-CYTE), fetuin, bovine serum albumin (BSA), serum albumin, and transferrin. Commercially available media such as Minimal Essential Medium (MEM, Sigma), Ham's F10 (Sigma), or Dulbecco's Modified Eagle's Medium (DMEM, Sigma) may be used as the serum-free medium in accordance with the invention. When serum free media is selected lipids such as, e.g., palmitic acid and/or steric acid, can be included.

Lipids components include oils, saturated fatty acids, unsaturated fatty acids, glycerides, steroids, phospholipids, sphingolipids and lipoproteins.

Exemplary amino acid that can be included or eliminated from the media include alanine, arginine, asparagine, aspartic acid, cysteine, glutamic acid, glutamine, glycine, histidine, proline, isoleucine, leucine, lysine, methionine, phenylalanine, proline, serine, threonine, tryptophan, tyrosine and valine.

Examples of vitamins that can be present in the media or eliminated from the media include vitamin A (retinoid), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid), vitamin B12 (cyanocobalamin), vitamin C (ascorbic acid), vitamin D, vitamin E, and vitamin K.

Minerals that can be present in the media or eliminated from the media include bismuth, boron, calcium, chlorine, chromium, cobalt, copper, fluorine, iodine, iron, magnesium, manganese, molybdenum, nickel, phosphorus, potassium, rubidium, selenium, silicon, sodium, strontium, sulfur, tellurium, titanium, tungsten, vanadium, and zinc.

Osmolality can be adjusted by adding salt to the media or having salt be produced as a byproduct as evaporation occurs during production.

Hormones include, for example, somatostatin, growth hormone-releasing factor (GRF), insulin, prolactin, human growth hormone (hGH), somatotropin, estradiol, and progesterone. Growth factors include, for example, bone morphogenic protein (BMP), epidermal growth factor (EGF), basic fibroblast growth factor (bFGF), nerve growth factor (NGF), bone derived growth factor (BDGF), transforming growth factor-α1 (TGF-α1), [Growth factors from U.S. Pat. No. 6,838,284 B2], hemin and NAD.

Examples of surfactants that can be present or eliminated from the media include Tween-80 and pluronic F-68.

Small molecules can include, e.g., butyrate, ammonia, non natural sugars, non natural amino acids, chloroquine, and betaine.

In some embodiments, ammonia content can be selected as a production parameter to produce a desired polypeptide or polypeptide isoform or the desired profile of polypeptide and/or polypeptide isoforms. Ammonia can be directly added to the culture and/or can be produced as a by product of glutamine or glucosamine.

Another production parameter is butyrate content.

In some embodiments, a component such as an enzyme, sugar and/or sugar precursors can be added to media or batch fed to cells to effect glycosynthesis. For example, enzymes and substrates such as sugar precursors can be added to the media or batch fed to cells to produce a desired polypeptide isoform or the desired profile of polypeptide and/or polypeptide isoforms. These methods can make use of monosaccharide substrates that are taken up by a cell, converted to “activated” monosaccharide substrates in vivo and incorporated into the expressed protein by the cell. The methods are amenable to any cell that can be manipulated to produce a desired glycoprotein. The cell can use, e.g., endogenous biochemical processing pathways or can be genetically engineered to convert, or process, the exogenously added monosaccharide into an activated form that serves as a substrate for conjugation to a target glycoprotein in vivo or in vitro.

Monosaccharides added to a polysaccharide chain can be incorporated in activated form. Activated monosaccharides, which can be added, include UDP-galactose, UDP-glucose, UDP-N-acetylglucosamine, UDP-N-acetylgactosamine, UDP-xylose, GDP-mannose, GDP-fucose, CMP-N-acetylneuraminic acid and CMP-N-acetylglycolylneuraminic acid. Other monosaccharide precursors that can be added to media or batch fed to cells include: N-acetylglucosamine, glucosamine, glucose, galactose, N-acetylgalactosamine, fructose, fucose, glucose-6-phosphate, mannose-6-phosphate, mannose-1-phosphate, fructose-6-phosphate, glucosamine-6-phosphate, N-acetylglucosamine-6-phosphate, N-acetylmannosamine, N-acetylneuraminic acid-6-phosphate, fucose-1-phosphate, ATP, GTP, GDP, GMP, CTP, CDP, CMP, UTP, UDP, UMP, uridine, adenosine, guanosine, cytodine, lactose, maltose, sucrose, fructose 1,6 biphosphate, 2 phosphoenol pyruvate, 2-oxaloacetate and pyruvate.

Activated forms of monosaccharides can be generated by methods known in the art. For example, galactose can be activated to UDP-galactose by several ways including: direct phosphorylation at the 1-position to give Gal-1-P, which can react with UTP to give UDP-galactose; Gal-1-P can be converted to UDP-galactose via uridyl transferase exchange reaction with UDP-glucose that displaces Glc-1-P. UDP-glucose can be derived from glucose by converting glucose to Glc-6-P by hexokinase and then either to Fru-6-P by phosphoglucose isomerase or to Glc-1-P by phosphoglucomutase. Reaction of Glc-1-P with UTP forms UDP-glucose. GDP-fucose can be derived from GDP-Man by reduction with CH₂OH at the C-6 position of mannose to a CH₃. This can be done by the sequential action of two enzymes. First, the C-4 mannose of GDP-Man is oxidized to a ketone, GDP-4-dehydro-6-deoxy-mannose, by GDP-Man 4,6-dehydratase along with reduction of NADP to NADPH. The GDP-4-keto-6-deoxymannose is the epimerized at C-3 and C-5 to form GDP-4-keto-6-deoxyglucose and then reduced with NADPH at C-4 to form GDP-fucose. Methods of obtaining other activated monosaccharide forms can be found in, e.g., Varki, A et al., eds., Essentials of Glycobiology, Cold Spring Harbor Press, Cold Spring Harbor, N.Y. (1999).

An activated monosaccharide can be incorporated into a polysaccharide chain using the appropriate glycosyltransferase. For example, to incorporate a sialic acid, CMP-sialic acid onto a polysaccharide chain, a sialyltransferase, e.g., É2→3 sialyltransferase or É2→6 sialyltransferase, can be used. To incorporate a fucose, a fucosyltransferase, e.g., É1→2 fucosyltransferse, É1→3 fucosyltransferase, É1→4 fucosyltransferase or É1→6 fucosyltransferase, can be used. Glycosyltransferases for incorporating galactose and GlcNAc include a galactosyltransferase (e.g., É1→3 galactosyltransferase, é1→4 galactosyltransferase or é1→3 galactosyltransferase) and a N-acetylglucosaminyltransferase (e.g., N-acetylglucosaminyltransferase I, II or III), respectively. Glycosyltransferases for incorporating other monsaccharides are known. The glycosyltransferase can be added to the media or batch fed to the cell or the cell can use endogenous processing pathways or be genetically engineered to convert or process the exogenously added monosaccharide.

Some aspects include having glucosamine present in the media. Glucosamine can be added to the media or batch fed to the cell or the appropriate enzymes and/or substrates can be added to the media or batch fed to cells such that glucosamine is produced. For example, one or more of N-acetylglucosamine, N-acetylglucosamine 6-phosphate, N-acetylmannosamine or fructose can be added to the media or batch fed to the cell for production of glucosamine.

The methods can further include having uridine added to the media or batch fed to a cell.

Methods described herein can include selecting culture conditions that are correlated with a desired process characteristic. Such conditions can include temperature, pH, osmolality, shear force or agitation rate, oxidation, spurge rate, growth vessel, tangential flow, DO, CO₂, nitrogen, fed batch, redox, cell density, perfusion culture and feed strategy.

For example, the production parameter can be culturing a cell under acidic, neutral or basic pH conditions. Temperatures can be selected from 10 to 42° C.

In other embodiments, carbon dioxide levels can be selected which results in a desired polypeptide and/or polypeptide isoform or the desired profile of polypeptide and/or polypeptide isoforms. CO₂ levels can be, e.g., about 5%, 6%, 7%, 8%, 9%, 10%, 11%, 13%, 15%, 17%, 20%, 23% and 25% (and ranges in between). CO₂ levels can be adjusted manually or can be a cell byproduct.

A wide array of flasks, bottles, reactors, and controllers allow the production and scale up of cell culture systems. The system can be chosen based, at least in part, upon its correlation with a desired process characteristic, e.g., the desired polypeptide and/or polypeptide isoform or the desired profile or polypeptide and/or polypeptide isoforms.

Cells can be grown, for example, as batch, fed-batch, perfusion, or continuous cultures as described in more detail below.

Production parameters that can be selected include, e.g., addition, removal or maintenance of media including when (early, middle or late during culture time) and how often media is harvested; increasing, decreasing or maintaining the speed at which cell cultures are agitated; increasing, decreasing or maintaining the temperature at which cells are cultured; adding or removing media such that culture density is adjusted; selecting a time at which cell cultures are started or stopped; and selecting a time at which cell culture parameters are changed. Such parameters can be selected for any of the batch, fed-batch, perfusion and continuous culture conditions, e.g., described below.

Batch Culture: Batch culture is carried out by placing the cells to be cultured into a fixed volume of culture medium and allowing the cells to grow. Cell numbers increase, usually exponentially, until a maximum is reached, after which growth becomes arrested and the cells die. This may be due either to exhaustion of a nutrient or accumulation of an inhibitor of growth. To recover product, cells are removed from the medium either when the cells have died or at an earlier, predetermined point. Batch culture is characterized in that it proceeds in a fixed volume (since nothing is added after placing the cells in the medium), for a fixed duration (dependent on the length of time the cells survive) with a single harvest and with the cells dying or being discarded at the end of the process.

Fed-Batch Culture: This is a variation on batch culture and involves the addition of a feed to the batch. Cells are cultured in a medium in a fixed volume. Before the maximum cell concentration is reached, specific supplementary nutrients are added to the culture. The volume of the feed is minimal compared to the volume of the culture. A fed-batch culture involves a batch cell culture to which substrate, in either solid or concentrated liquid form, is added either periodically or continuously during the period of growth. Fed batch culture is also characterized in that it usually proceeds in a substantially fixed volume, for a fixed duration, and with a single harvest either when the cells have died or at an earlier, predetermined point. Fed-batch cultures are described, e.g., in U.S. Pat. No. 5,672,502.

Perfusion Culture: In a perfusion culture, medium is perfused through the reactor at a high rate while cells are retained or recycled back into the reactor by sedimentation, centrifugation or filtration. Up to ten reactor volumes of medium is perfused through the bioreactor in a day. The major function of perfusing such a large volume of medium is primarily to remove the metabolites, mainly lactate, from the culture fluid. Perfusion cultures are described, e.g., in U.S. Pat. No. 6,544,788.

Continuous Culture: In continuous culture, the cells are initially grown in a fixed volume of medium. To avoid the onset of the decline phase, a pumped feed of fresh medium is initiated before maximum cell concentration is reached. Culture, containing a proportion of the cells, is continuously removed from the vessel to maintain a constant volume. The process removes a product, which can be continuously harvested, and provides a continuous supply of nutrients, which allows the cells to be maintained in an exponentially growing state. A continuous culture is characterized by the continuous maintenance of an exponentially growing culture. There is little or no death or decline phase. In a continuous culture, cells are continuously fed fresh nutrient medium, while spent medium, cells, and excreted cell product are continuously drawn off. Continuous cultures and bioreactors are described, e.g., in U.S. Pat. Nos. 4,764,471; 5,135,853; and 6,156,570.

### Bioreactors

A bioreactor is a device or system that supports a biologically-active environment, e.g., a device or system meant to grow cells or tissues in the context of cell culture (e.g., mammalian, plant, yeast, bacterial cells). This process can either be aerobic or anaerobic. Bioreactors are commonly cylindrical, ranging in size from some liter to cube meters, and are often made of stainless steel. On the basis of mode of operation, a bioreactor may be classified as batch, fed batch or continuous (e.g. continuous stirred-tank reactor model).

A bioreactor can be used for large culture volumes (in the range 100-10,000 liters). Suspension cell lines can be kept in suspension, e.g., by a propeller in the base of the chamber vessel (e.g., stir tank or stir flask bioreactors) or by air bubbling through the culture vessel. Both of these methods of agitation can give rise to mechanical stresses. Membranes, porous matrices (e.g., ceramic matrices), and polysaccharide gels can be used to protect cells from shear and/or to obtain high cell densities in bioreactors that are productive for periods of weeks or months.

Rotary bioreactors use rolling action to keep cells well perfused, akin to roller bottles. In order to create a high-density environment, the culture chamber can be separated from the feeder chamber by a semipermeable membrane. This allows media to be changed without disturbing the cells. Using this principle, the rotating action in Synthecon's Rotary Cell Culture System (RCCS; Houston, Tex.) creates a microgravity environment, virtually eliminating shear forces. This allows the cell to shift resources from damage control to establishing relationships with other cells, mimicking the complex three-dimensional (3-D) matrices found in vivo. Reactor vessels come in sizes ranging from 10 mL to 500 mL.

Non-limiting examples of bioreactors are as follows.

The Heraeus miniPERM bioreactor combines an autoclavable outer nutrient container and a disposable inner bioreactor chamber. The appropriate molecular weight cut-off membrane for a desired product (e.g., a product described herein) can be selected. Its small size allows it to fit inside standard incubators. Densities greater than 10⁷ cells per mL and product yields of 160 mg in four weeks are possible.

New Brunswick Scientific's CELLIGEN PLUS® (Edison, N.J.) is a highly flexible system for culture of virtually all eukaryotic cell lines. Features include a double screen impeller for increased O₂ saturation, interactive four-gas control, internal ring sparger, five programmable pumps, computer interface for system control and data logging, and four-channel recorder output. The unit may be used either as a stir tank or fibrous-bed system.

The Wave BIOREACTOR™ (from Wave Biotech, LLC, Somerset, N.J.) employs an adjustable-speed rocking platform and electric air pump to gently aerate the culture while keeping shear forces low. Smaller cultures and rocking platforms will fit in a standard incubator. Culture medium and cells only contact a presterile, disposable chamber called a cellbag that is placed on a special rocking platform. The rocking motion of this platform induces waves in the culture fluid. These waves provide mixing and oxygen transfer, resulting in a perfect environment for cell growth that can easily support over 20×10⁶ cells/mL. The bioreactor requires no cleaning or sterilization, providing the ultimate ease in operation and protection against cross-contamination.

Quark Enterprises (Vineland, N.J.) provides a full range of bioreactors including its SPINGRO® flasks for high-density culture. These borosilicate stir flasks range from 100 mL to 36 L and feature TEFLON® spin paddles, side vents for probes and easy sampling, and jacketed models for use with a recirculating water bath. All models are completely autoclavable.

The ProCulture DynaLift system (Corning; Corning, N.Y.) facilitates perfusion and reduces shear effects by using an extended paddle, side baffles, and bottom contours. It is available in a range of sizes, from 125 mL to 36 L.

Another example is Braun Biotech's BIOSTAT® Bioreactor.

The largest cultures of cells have often been achieved in fermenter-type systems. Suspension cells are most direct to scale up in this system. Cell growth and harvesting is often straightforward once the parameters for achieving maximum product have been delineated. In-line monitors for pH, gas saturation, and metabolites are available from most suppliers. Adherent cells pose more of a challenge. Some can be “suspension-adapted.” Microcarrier beads as a support can be employed to improve culturing (see below).

### Stir Tank Bioreactors

Stir tanks (and flasks) can provide cell cultures with increased density. Examples include the following.

A disposable Stirred Tank Bioreactor (Xcellerex): a scaleable, disposable stir tank bioreactor (XDR™) that can operate as a stand-alone skid mounted system or is integrated into a FLEXFACTORY™. The XDR incorporates process sensors that monitor and control the culture conditions up to 1,000 L or 2,000 L working volume scale. FLEXFACTORY™ is a complete, turnkey, modular production train for biotherapeutics and vaccines. The single-use, disposable components that are central to the FLEXFACTORY™, provide it with great flexibility to accommodate new process changes, including production of multiple products at a single site, and to establish manufacturing capacity rapidly, at dramatically lower costs than traditional fixed-tank, hard-piped facilities.

Applikon offers a full line of stir tanks, from 2.3 L bench systems to 10,000 L production units. Pumps, probes, controllers, and software are also available for all units. Borosilicate glass vessels are available up to 20 L and can be fitted with lip-sealed or magnetically coupled stirrers. Stainless steel BIOCLAVE™ vessels are designed for moderate to large-scale production and feature a flush-mounted longitudinal sight glass as well as a choice of lip-seal or magnetic stirrers.

### Airlift Bioreactors

An alternative to the stirred tank is the airlift bioreactor. The reactor has no moving blades to create shear forces, which some mammalian and hybridoma cells are particularly sensitive to. Media perfuse from the top while oxygen enters through the bottom, creating a near-ideal mixing environment.

Kimble-Kontes (Vineland, N.J.) manufactures the CYTOLIFT® glass airlift bioreactor with an effective volume of 580 mL. It is easily cleaned and fully autoclavable for consistent performance and long life. A glass jacket is standard on all models. Other features include a check valve to prevent backflow in case of pressure drop, vent, infusion and effluent ports, plus three ports for pH, foam level, and dissolved oxygen (dO2) probes. CYTOSTIR® (also from Kimble-Kontes) is a line of double-sidearm bioreactors in nine sizes, from 100 mL to 36 L. The large, height-adjustable stirring blades are constructed of TEFLON® to minimize cell adhesion and facilitate cleaning. Components are steam-autoclavable.

### Batch Bioreactors

In batch bioreactors, the medium and inoculum are loaded in the beginning and the cells are allowed to grow. There is no addition/replacement of medium, and the entire cell mass is harvested at the end of incubation period. The characteristic features of such bioreactor systems are as follows: (i) continuous depletion of medium, (ii) accumulation of cellular wastes, (iii) alterations in growth rate and (iv) continuous change in the composition of cells.

A spin filter bioreactor can be used as a batch bioreactor by closing the inlet for medium and the outlets for medium/medium plus cells.

Batch bioreactors are available, e.g., from Rockland Immunochemicals, Inc.

### Fed-Batch Bioreactors

In fed-batch (semi-batch) reactors, feed is added, but effluent (and cells) are not removed. Thus fed-batch reactors can be used to maintain cells under low substrate or nutrient conditions without washout occurring. Because cells are not removed during the culturing, fed-batch bioreactors are well suited for the production of compounds produced during very slow or zero growth. Unlike a continuous bioreactor, the feed does not need to contain all the nutrients needed to sustain growth. The feed may contain only a nitrogen source or a metabolic precursor.

### Continuous Bioreactors

In continuous bioreactors, there is continuous inflow of fresh medium and outflow of used medium (with or without cells) during the entire incubation period. The cells thus continuously propagate on the fresh medium entering the reactor and at same time, products, metabolic waste products and cells are removed in the effluent. A spin filter bioreactor is an example of continuous flow bioreactor. It can have the following features: (1) The central shaft of bioreactor houses a spinning, filter, which enables the removal of used medium, free of cells, through the shaft; (2) A stirrer plate magnetically coupled to the central shaft provides continuous stirring; the spinning filter also stirs the culture; (3) The culture is aerated by a sparger, which allows a wide range of aeration rates; (4) A port is provided for addition of fresh medium, while (5) Another port enables removal of the culture (used medium+cells) as need.

This bioreactor provides a highly versatile system for control on medium change rate and on cell density; this becomes possible due to the two routes for medium removal, while only one of them allows the removal of cells.

A continuous flow bioreactor can be used to grow cells at a specified cell density in an active growth phase; such cultures may either provide inocula for further culture or may serve as a continuous source of biomass yields.

### Immobilized Cell Bioreactors

These bioreactors are based on cells entrapped either in gels, such as, agarose, agar, chitosan, gelatine, gellan, polyacrylamide and calcium alginate, to produce beads, or in a membrane or metal (stainless steel) screen compartment or cylinder.

As an example of the operation of such a bioreactor: the membrane screen cylinder containing cells is kept in a chamber through which the medium is circulated from a recycle chamber. The medium flows parallel to the screen cylinder and diffuses across the screen into the cell mass.

Similarly, products from cells diffuse into the medium and out of the screen cylinder. The membrane/screen compartment housing the cells may be cylindrical or flat, and medium movement may be so adjusted as to flow across the screen compartment rather than parallel to it. Fresh medium is regularly added to and equivalent volume of used medium is withdrawn from the recycling chamber to maintain its nutrient status.

Cell immobilization changes the physiology of cells as compared to that of cells in suspension. This technique is useful where the biochemical of interest is excreted by the cells into the medium.

Product excretion may also be brought about by immobilization itself, or by certain treatments like altered pH, use of DMSO (dimethyl sulfoxide) as a permeabilizing agent, changed ionic strength of medium, an elicitor, etc.

Immobilized cell reactors can have the following advantages: (i) no risk of cell wash out, (ii) low contamination risk, (iii) protection of cells from liquid shear, (iv) better control on cell aggregate size, (v) separation of growth phase (in a batch/continuous bioreactor) from production stage (in an immobilized cell bioreactor), (vi) cellular wastes regularly removed from the system, and (vii) cultures at high cell densities.

### Multistage Bioreactors

Such culture systems use two or more bioreactors in a specified sequence, each of which carries out a specific step of the total production process. The simplest situation would involve two bioreactors. For example, for the production of a biochemical, both the bioreactors can be of batch type: the first bioreactor provides conditions for rapid cell proliferation and favors biomass production, while the second bioreactor has conditions conducive for biochemical biosynthesis and accumulation. The cell biomass is collected from the first stage bioreactor and is used as inoculum for the second stage reactor. As another example, the first reactor may be in continuous mode, while the second may be of batch type.

The cell mass from this bioreactor serves as a continuous source of inoculum for the second stage batch type bioreactor, which has conditions necessary for embryo development and maturation (but not for cell proliferation). The use of continuous first stage bioreactor can offer one or more advantages, e.g.: (i) avoids the time, labor and cost needed for cleaning, etc. of a batch reactor between two runs, (ii) eliminates the lag phase of batch cultures, and (iii) provides a more homogeneous and actively growing cell population.

### Perfusion Bioreactors

Bioreactors are available for perfusion cultures. Examples are as follows.

The CELLCUBE® System from Corning Life Sciences provides a fast, simple, and compact method for the mass culture of attachment dependent cells in a continuously perfused bioreactor. The system is an easily expandable system for growing adherent cells in all levels of biomass, viral, and soluble biomolecule production. The basic system uses disposable CELLCUBE® Modules with from 8,500 cm² to 85,000 cm² cell growth surface using the same control package. CELLCUBE® Modules have polystyrene growth surfaces that are available with either the stand tissue culture surface or the advanced CORNING CELLBIND® Surface for improved cell attachment. These disposable polystyrene modules hold 3.5 l of media and contain 25 parallel plates for a total growing area of 21,000 cm2 per cube, expandable up to 340,000 cm2 (the 4/100 stack). The interlinkable cubes stand on one corner with media entering the bottom and exiting the top. The CELLCUBE® System is comprised of four pieces of capital equipment—the system controller, oxygenator, circulation and media pumps. The digital controller features in-line monitoring of perfusion, pH, dO₂, and temperature.

### Centrifugal Bioreactors

Another type of bioreactor is a centrifugal bioreactor, e.g., Kinetic Biosystems' CBR 2000 centrifugal bioreactor. Designed for industrial production, it can achieve densities up to 10,000 times greater than stirred tank bioreactors. Media are fed in through the axle, then forced to the outside by the rotating action where they enter the reaction chamber. Cells are held in suspension by opposing centrifugal force with perfusion. Waste products are removed through the axle and sampled 10 times per hour. Real-time analysis of growth and production parameters means that any perturbation can be adjusted quickly. The end result can be increased product yield and quality. Each chamber is capable of producing 1×10¹⁶ cells with each rotor holding three chambers.

### Microcarrier

For attached cell lines (e.g., for bioreactor cell culturing), the cell densities obtained can be increased by the addition of micro-carrier beads. These small beads are 30-1005 μm in diameter and can be made, e.g., of dextran, cellulose, gelatin, glass or silica, and can increase the surface area available for cell attachment. The range of micro-carriers available means that it is possible to grow most cell types in this system.

Particles come in two forms: solid and porous. Solid beads are the most manageable for biomass harvest, while porous beads are better suited for secreted or lysate products. Other matrices hold beads stationary, creating a solid bed through which media are perfused.

Microcarrier cultures using suspended macroporous beads are readily scaled up. These systems are distinct from conventional surface microcarrier culture in that the cells are immobilized at high densities inside the matrix pores and are protected from the fluidshear. Another advantage of macroporous beads is that they can be inoculated directly from the bulk medium in the same fashion as conventional microcarriers. Suspended bead immobilization systems can be used in a number of different reactor configurations including suspended beds or stirred tank bioreactors. These systems can be scaled up by increasing the volume of the bioreactor and the number of beads. Suspended macroporous bead technologies are also available. In an attempt to mimic the cell culture environment in mammals, these macroporous beads can be collagen-based (e.g., collagen, gelatin, or collagen-glycosaminoglycan).

For example, Porous ImmobaSil microbeads produced by Ashby Scientific are available in different shapes and sizes for easy adaptation to your particular culture vessel. They are gas permeable, allowing culture densities to reach 3×10⁶/mL for maximum product yield.

Amersham Pharmacia (AP) Biotech offers microcarriers and fluid-bed reactors. Cytopore I beads are optimized for CHO-type cells, while Cytopore II is for adherent cells requiring higher surface-charge density. Cytoline I beads are suited for resilient cells requiring high circulation rates. The low-density Cytoline II carrier is optimized for shear-sensitive cells such as hybridomas needing slower circulation. AP Biotech has designed the Cytopilot fluid-bed system perfusion reactor for use with its Cytoline beads.

Glass-surface microcarrier for growth of cell cultures are described in U.S. Pat. No. 4,448,884.

Further, the CYTOSTIR® line (from Kontes) of double sidearm stirred bioreactors for microcarrier cell culture has been completely redesigned to improve performance and enhance interchangeability. CYTOSTIR® bioreactors are available in nine sizes ranging from 100 mL to 36 liters. The borosilicate glass flasks have two large sidearms with screw cap closures that allow easy sampling. The dome in the center of the flask base prevents microcarriers from accumulating directly under the stirring blades. The large, height adjustable TEFLON® stirring blades are designed to provide maximum stirring efficiency to keep microcarriers in suspension at the slow stirring speeds required for tissue culture. During stirring, cultures contact only borosilicate glass and TEFLON®. All one liter and larger size flasks have anti-drip pour lips and polypropylene caps with sealing rings. All CYTOSTIR® bioreactors and components are completely steam autoclavable.

### Spinner Culture

This is a common culture method for suspension lines including hybridomas and attached lines that have been adapted to growth in suspension. Spinner flasks are either plastic or glass bottles with a central magnetic stirrer shaft and side arms for the addition and removal of cells and medium, and gassing with CO₂-enriched air. Inoculated spinner flasks are placed on a stirrer and incubated under the culture conditions appropriate for the cell line. Cultures can be stirred, e.g., at 100-250 revolutions per minute. Spinner flask systems designed to handle culture volumes of 1-12 liters are available from Techne, Sigma, and Bellco, e.g. (Product No. Z380482-3L for a 3 liter capacity and Product No. Z380474-1 L for a 1 liter capacity). Another example of spinner culture systems is the MantaRay single-use spinner flask.

Wheaton Science Products offers scale-up systems for all levels of production. Its MAGNA-FLEX® Spinner Flasks have bulb-shaped, flex-type glass impellers for use with microbeads. A removable stainless steel pin immobilizes the impeller to prevent cell damage during handling. Available in a range of sizes from 125 mL to 8 L, they are fully autoclavable. Also available are the CELL OPTIMIZER™ System for determination of optimum culture conditions prior to scale-up, and the OVERDRIVE™ for economical industry-level production up to 45 l.

The SuperSpinner from B. Braun Biotech is an entry-level stir flask that accommodates 500 and 1000 mL cultures and features a bubble-free aeration/agitation system. The BIOSTAT® series of stir vessels handles culture sizes from 50 mL to 10 L and include complete ready-to-use systems and systems that integrate preexisting components.

Techne UK offers a complete line of stir flasks in volumes up to 5 l. Designed with a stirring rod rather than paddles, they simplify cleaning and autoclaving by eliminating rotating bearings. The unique stirring action creates vertical and horizontal flow in a gentle spiral throughout the culture. Its line of programmable stirring platforms features the SOFTSTART™ acceleration/deceleration control to reduce cell damage from excessive turbulence.

Wheaton Science Products offers scale-up systems for all levels of production. Its MAGNA-FLEX® Spinner Flasks have bulb-shaped, flex-type glass impellers for use with microbeads. A removable stainless steel pin immobilizes the impeller to prevent cell damage during handling. Available in a range of sizes from 125 mL to 8 L, they are fully autoclavable. Also available are the CELL OPTIMIZER™ for determination of optimum culture conditions prior to scale-up, and the OVERDRIVE™ for economical industry-level production up to 45 l.

### T Flask Culture

Adherent or suspension cultures can be grown in T flasks, e.g., T-25, T-76, T-225 flasks. The caps can be plug sealed or vented. The flasks can be plastic or glass. The surface of the flasks can be coated, e.g., with hydrophilic moieties that contain a variety of negatively charged functional groups and/or nitrogen-containing functional groups that support cell attachment, spreading, and differentiation. T flasks are available, e.g., from Nunc, Nalgene, Corning, Greiner, Schott, Pyrex, or Costar.

### Cell Culture Dishes

Cells can be grown in culture dishes. The surface of the dishes can be coated, e.g., with hydrophilic moieties that contain a variety of negatively charged functional groups and/or nitrogen-containing functional groups that support cell attachment, spreading, and differentiation. Dishes are available, e.g., from BD Biosciences, Corning, Greiner, Nunc, Nunclon, Pyrex.

### Suspension Cell Culture

Suspended cells can be grown, e.g., in bioreactors, dishes, flasks, or roller bottles, e.g., described herein.

### Stationary Suspension Culture Systems

An example of a stationary suspension system is CELLine™ 1000. The CELLine™ 1000 (Integra Bioscience, Chur, Switzerland) device is a membrane-based disposable cell culture system. It is composed of two compartments, a cultivation chamber (20 mL) and a nutrient supply compartment (1000 mL) separated by a semipermeable dialysis membrane (10 kD molecular weight cut-off), which allows small nutrients and growth factors to diffuse to the production chamber. Oxygen supply of the cells and CO₂ diffusion occur through a gas-permeable silicone membrane. Antibodies concentrate in the production medium. This culture system requires a CO₂ incubator. For example, for optimal production levels, the device can be inoculated with 50×10⁶ cells, and 80% of the production medium and the entire nutrition medium changed twice a week.

### Rotation Suspension Culture Systems

Such systems include roller bottles (discussed herein). An example of a rotation suspension system is the miniPERM (Vivascience, Hannover, Germany), which is a modified roller bottle two-compartment bioreactor in which the production module (35 mL) is separated from the nutrient module (450 mL) by a semipermeable dialysis membrane. Nutrients and metabolites diffuse through the membrane, and secreted antibodies concentrate in the production module. Oxygenation and CO₂ supply occur through a gas-permeable silicone membrane at the outer side of the production module and through a second silicone membrane extended into the nutrition module. The miniPERM must be placed on a roller base inside a CO₂ incubator. It is possible to place two roller bases together in a 180-L CO2 incubator, each holding a maximum of four bioreactors (i.e., the same amount of space is occupied for 1-4 incubations).

### Roller Bottle

This is the method most commonly used for initial scale-up of attached cells also known as anchorage dependent cell lines. Roller bottles are cylindrical vessels that revolve slowly (between 5 and 60 revolutions per hour) which bathes the cells that are attached to the inner surface with medium. Roller bottles are available typically with surface areas of 1050 cm2 (Prod. No. Z352969). The size of some of the roller bottles presents problems since they are difficult to handle in the confined space of a microbiological safety cabinet. Recently roller bottles with expanded inner surfaces have become available which has made handling large surface area bottles more manageable, but repeated manipulations and subculture with roller bottles should be avoided if possible. A further problem with roller bottles is with the attachment of cells since as some cells lines do not attach evenly. This is a particular problem with epithelial cells. This may be partially overcome a little by optimizing the speed of rotation, generally by decreasing the speed, during the period of attachment for cells with low attachment efficiency.

Roller bottles are used in every conceivable application. A good starting point for small labs with periodic scale-up needs, they are also being used for large-scale industrial production. Because the cultures are seeded and maintained in a manner similar to flasks, typically no additional training is necessary. Small racks fit inside standard incubators, eliminating the need for additional capital expenditures.

Roller bottles come in a number of configurations: plastic, glass, pleated, flat, vented, or solid. Glass can be sterilized and reused, whereas different plastics and coatings optimize growth for an assortment of cell types. Pleats increase the effective growth surface, thereby increasing product yield without additional space requirements. Vented caps are used for culture in a CO₂ environment, while solid caps are best for culturing in a warm room or unregulated incubator. Roller bottles are available, e.g., from Corning.

### Adherent Cell Culture

Adherent cells can be grown, e.g., in bioreactors, dishes, flasks, or roller bottles, e.g., described herein. The surfaces to which the cells adhere can be treated or coated to promote or support cell attachment, spreading, and/or differentiation. Coatings include lysine (e.g., poly-D-lysine), polyethyleneimine, collagen, glycoprotein (e.g., fibronectin), gelatin, and so forth.

### Shaker Flask

Shaker flasks can be used to provide greater agitation of cell cultures to improve oxygen or gas transfer, e.g., as compared to stationary cultures. Shaker flasks are available, e.g., from Pyrex and Nalgene.

### Perfusion

Perfusion systems allow for continuous feeding of the cell chamber from external media bottle, as described herein. Cells are retained in the cell chamber (e.g., bioreactor, bed perfusion bioreactor, packed bed perfusion bioreactor). Suppliers of perfusion systems include DayMoon Industries, Inc., and New Brunswick Scientific.

### Hollow Fiber Cell Culture

Hollow fibers are small tube-like filters with a predefined molecular weight cutoff. Large bundles of these fibers can be packed into cylindrical modules, which provide an absolute barrier to cells and antibodies while ensuring perfusion of the liquid. Hollow fiber modules can provide a large surface area in a small volume. The walls of the hollow fibers serve as semipermeable ultrafiltration membranes. Cells are grown in the extracapillary space that surrounds the fibers, and medium is perfused continuously inside the fibers. Metabolites and small nutrients freely perfuse between extra- and intracapillary space according to concentration gradients. Culture monitoring can be performed by lactate measurement.

Example of such systems include: Cellex Biosciences' AcuSyst hollow fiber reactor.

Another example is the CELL-PHARM® system 100 (CP100, BioVest, Minneapolis, Minn.) which is a fully integrated hollow fiber cell culture system. The cell culture unit consists of two cartridges: one that serves as a cell compartment and the other, as an oxygenation unit. The system is a freestanding benchtop system with a disposable flowpath with yields of up to 400 mg/month.

The CELL-PHARM® system 2500 (CP2500, BioVest) is a hollow fiber cell culture production system that can produce high-scale quantities of a cell-produced product, e.g., of monoclonal antibodies. Unlike CP100, it consists of two fiber cartridges for the cells and hence offers a large cell growth surface (3.25 m2). A third cartridge serves for oxygenation of the medium.

The FIBERCELL™ (Fibercell Systems Inc., Frederick, Md.) hollow-fiber cell culture system is composed of a culture medium reservoir (250 mL) and a 60-mL fiber cartridge (1.2 m2), both connected to a single microprocessor-controlled pump. It is possible to prolong the media supply cycles by replacing the original medium reservoir with a 5-L flask. In contrast to the CELL-PHARM® systems, the FIBERCELL™ bioreactor is used inside a CO₂ incubator. Oxygenation occurs by a gas-permeable tubing.

Cellex Biosciences makes hollow fiber reactors for all levels of production. The ACUSYST-XCELL® is designed for large-scale production of secreted proteins, producing 60 to 200 grams of protein per month. Its ACUSYST MINIMAX™ is a flexible research scale benchtop bioreactor capable of producing up to 10 g of protein per month. For single-use or pilot studies of a few weeks' duration, the economical RESCU-PRIMER™ produces up to 200 mg per month with a choice of hollow fiber and ceramic matrices.

The Unisyn CELL-PHARM® MICROMOUSE™ is a disposable system with a footprint of 1.5 ft2, fitting inside a standard lab incubator. It is capable of producing up to 250 mg of monoclonal antibodies per month for three months.

The TECNOMOUSE® by Integra Biosciences is a modular rack system with five separate cassette chambers. Up to five different cell lines can be cultivated for up to 30 weeks, each producing 200 mg of antibodies per month. The integrated gas supply and online monitoring capabilities help to control culture conditions.

### Cell Factories

Cell factories are used for large scale (e.g., industrial scale) cell culture and products of biomaterials such as vaccines, monoclonal antibodies, or pharmaceuticals. The factories can be used for adherent cells or suspension culture. The growth kinetics are similar to laboratory scale culture. Cell factories provide a large amount of growth surface in a small area with easy handling and low risk of contamination. A cell factory is a sealed stack of chambers with common vent and fill ports. A 40-chamber factory can be used in place of 30 roller bottles. Openings connecting the chambers cause media to fill evenly for consistent growth conditions. Vents can be capped or fitted with bacterial air vents. Cell factories can be molded from e.g., polystyrene. Suppliers of cell factories include Nunc. The surface of the factories can be treated to improve growth or cell attachment conditions, e.g., treated with NUNCLON®Δ.

NUNCLON® CELL FACTORIES™ are low-profile, disposable, polystyrene, ventable chambers that come in stacks of one, two, 10, and 40. Inoculation, feeding, and harvest are straightforward due to the innovative design of the connected plates.

### Cell Culture Bags

Cell culture bags, e.g., single use cell culture bags, can be used for growing mammalian, insect, and plant cells. The bags present convenience and flexibility for suspension, perfusion, and microcarrier culture. Suppliers of cell culture bags include DayMoon Industries, Inc. and Dunn Labortechnik GmbH.

Gentle wave motion induced by agitation of the bags creates an excellent mixing and oxygenation environment for cell growth. Equipped with internal dip-tube and mesh filter, media exchange and perfusion culture with microcarriers is simplifies. A built-in screw-cap port can provide convenience for unrestricted access of microcarrier beads, cell attachment matrix and tissue cultures.

The bag system also offers a greater flexibility in gas transfer between the bag headspace and the environment, and it is capable of both gas diffusion and continuous gas flush. Gas diffusion through the built-in microporous membrane on the screw-cap provides sufficient gas exchange for most cell culture need. If required, pressurized air or gas under 1.5 psi can be added through one of the luer ports and vented out through the membrane cap.

As an example, OPTIMATA™ is a single-use cell culture bag that offers convenience, capacity and flexibility for growing insect, plant and mammalian cells. OPTIMA™ is designed for use on conventional laboratory shakers or rocking platforms. Available in two standard bags with working capacities up to 4 l, the OPTIMA™ is useful for high volume suspension culture, providing a cost-effective alternative to stirred bioreactors. OPTIMA-MINI™ bags are designed to fit most laboratory shakers and rocking platforms, requiring no specialized equipment.

### Protein Purification

The methods of the invention can be used to evaluate the effect, if any, of varying isolation or purification conditions on a process characteristic of a polypeptide preparation. In certain embodiments, an expressed polypeptide is secreted into the medium and thus cells and other solids may be removed, as by centrifugation or filtering for example, as a first step in the purification process.

In some embodiments, an expressed protein is bound to the surface of the host cell. In such embodiments, the media is removed and the host cells expressing the polypeptide or protein are lysed as a first step in the purification process. Lysis of mammalian host cells can be achieved by any number of means known to those of ordinary skill in the art, including physical disruption by glass beads and exposure to high pH conditions.

A protein may be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997), each of which is incorporated herein by reference in its entirety). For immunoaffinity chromatography in particular, the protein may be isolated by binding it to an affinity column comprising antibodies that were raised against that protein and were affixed to a stationary support. Affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin may be added at any or all stages in order to reduce or eliminate degradation of the polypeptide or protein during the purification process. Protease inhibitors are particularly advantageous when cells must be lysed in order to isolate and purify the expressed polypeptide or protein.

### Application for the Analysis

Data has been generated by the methods described herein can be used in a variety of applications including the applications described below.

Information obtained from the methods described herein can be used to quantitatively and qualitatively determine one or more process characteristics of recombinant polypeptides, thus providing, e.g., titer and/or product-related species information for a bioreactor process. In certain embodiments, where polypeptides produced in serum-free media are evaluated for process characteristics, a sample of culture medium is conditioned prior to analysis by liquid chromatography, e.g., reverse-phase LC, size exclusion LC, normal phase LC, hydrophobic interaction LC, anion exchange LC, weak cation exchange LC, and similar chromatographic techniques can also be used. Preferably, the aforementioned LC forms are a high performance LC. In one embodiment, the cell culture sample is conditioned by centrifugation and filtered through a 0.22 μm filter. Remarkably, the methods do not require any prior chromatography steps (e.g., affinity chromatography) to evaluate a process characteristic by liquid chromatography. Therefore, the methods described herein have application for multiple recombinant polypeptide products and multiple polypeptides produced and secreted in a serum-free manufacturing process such as those with titers above 0.007 μg/μL. The method can be interfaced with automated sampling technologies for on-line monitoring with near-real-time analysis of polypeptide production.

The methods described herein can also be used to quantitatively determine concentrations of vitamins in a cell culture media sample, e.g., taken from a process bioreactor. In certain embodiments where vitamin levels are evaluated, cell culture samples are conditioned prior to analysis by reverse phase ultra high performance liquid chromatography. In one embodiment, the sample centrifuged and filtered through a 0.22 μm filter prior to evaluation by reverse-phase ultra high performance liquid chromatography.

When determining bioreactor condition for new cell lines, the methods described herein can be used to provide nutritional conditions for which cell growth and health can be optimally maintained. These conditions can lower cell stresses in culture allowing cells to grow more rapidly and maintain health over longer periods of time during the bioreactor process cycles. Also by maintaining adequate vitamin levels, vitamins carried through the purification process are greatly reduced.

Methods described herein provide analytical information that can be provided to meet a regulatory requirement such as Process Analytical Technology (PAT) applications. Information that can be provided includes the amount (or titer) and product quality of recombinant polypeptides, the amount of vitamins in a process bioreactor sample, a characteristic chromatographic profile of the product-related components present in a bioreactor process sample, the quantitative relationship of components present in a bioreactor process sample, and time-based process snapshots.

PAT is defined as “a system for designing, analyzing, and controlling manufacturing through timely measurements (i.e., during processing) of critical quality and performance attributes of raw and in-process materials and processes with the goal of ensuring final product quality.” PAT applications for the methods described herein include either on-line monitoring, when coupled with ex situ, automated bioreactor sampling technologies or at-line monitoring configurations. The information attained using the disclosed methods for PAT applications can provide the control of commercial manufacturing process and, e.g., provide assessment of product quality based on near-real-time measurements. Exemplary information that can be obtained by the disclosed methods includes process titers, isoform (glycoform) ratios, process events, or specific primary sequence or post-translational modification, which may effect the products activity or safety. The information obtained by the disclosed methods can be used to make decisions, such as: when to alter, feed, or stop a manufacturing process to maximize the productivity and product quality or quickly evaluating the impact of an errant manufacturing process parameter on product quality. Also, the methods can be applied to the monitoring of purification process steps for the on- or at-line monitoring of the product's process quality during this part of manufacturing. Finally, the ability to monitor a manufactured process and evaluate the manufactured product on- or at-line reduces the need for downstream characterization efforts.

The methods described herein can be used to define the comparability of both the manufacturing process and the expressed recombinant polypeptide. This information can be used to make process improvements for clinical and commercial manufacturing processes. Process improvements can include a change of the host cell line, enhancement of the cell culture medium, or improved cell culture management. In addition to establishing product comparability, methods described herein can be used to demonstrate that process impurities or byproducts are similar in different manufacturing processes, thus indicating that process changes have not altered the quality of the manufacturing process. A successful demonstration of comparability can accelerate the implementation of changes to a manufacturing process. The methods described herein can also be used as part of a comparison process of both the manufacturing process and exhausted feed nutrients.

During process development, the methods described herein can be used to obtain information about the product quality of the expressed polypeptide directly from the cell culture medium, without any sample manipulation or speciation prior to analysis that can be used for selecting a manufacturing process. Product quality endpoints obtained using the methods described herein include primary structure determination and isoform profiling of the polypeptide product, as well as other protein products that are expressed and secreted into the cell culture medium during the manufacturing process. Additionally, the information obtained can be used to make decisions about the quality of the cell line, the manufacturing processes, and the final product.

During the development of new polypeptide constructs and manufacturing processes, the methods disclosed herein can be used to rapidly determine the quality of the protein product and manufacturing process imparted by these new advances. Useful applications of the methods in this context can include determining the ratios of secreted polypeptides for various processes or polypeptide construct, clone and cell line selection and optimization, and cell culture management. Examples of cell culture management include type of culture (fed batch, perfusion, etc.), cell culture medium components, feed strategy, process length, and temperature shifts. In addition, the methods described herein can be used to analyze host cell proteins, after removal of product related proteins, and intercellular product-related and host cell proteins, after cell lyses, (+/−) Protein A and Protein L affinity capture, and concentration. These analyses provide information that is useful for defining, optimizing, and selecting both cell lines and cell culture processes, as well as providing data endpoints for technology development.

The methods described herein can also be used to characterize biopharmaceutical products in drug substance, with increased throughput.

One of ordinary skill in the art will appreciate that the exact purification technique may vary depending on the character of the polypeptide or protein to be purified, the character of the cells from which the polypeptide or protein is expressed, and/or the composition of the medium in which the cells were grown.

### EXAMPLES

### Example 1

### General Methods

Reagents. All solvents used in Liquid Chromatography (LC) and Mass Spectrometry (MS) experiments were of the highest quality and were used without further purification. HPLC grade acetonitrile was obtained from Burdick & Jackson (Honeywell International, Inc., Muskegon, Mich.). Ultrapure water was obtained using a Purelab Plus UV purification system (Type PL5112 02, US Filter, Lowell, Mass.). High purity (>99.5%) trifluoroacetic acid (TFA) was obtained from Pierce Biotechnology (Rockford, Ill.). Mass spectrometry grade formic acid was obtained from Fluka (Sigma-Aldrich Co., St. Louis, Mo.). Sodium iodide (NaI), bovine insulin, and bovine trypsinogen were obtained from Sigma (Sigma-Aldrich Co., St. Louis, Mo.).

Samples. All drug substance (DS), cell culture medium, and bioreactor process samples containing recombinant mAbs were produced by Wyeth BioPharma Development (Wyeth Biotech, Andover, Mass.). DS samples were manufactured in intermediate-scale manufacturing facilities and were stored in formulation buffer at −20° C. Prior to injection on-column, each DS standard was diluted with 0.05% TFA in water to 0.20 μg/mL via a two-step dilution. Bioreactor process or cell culture medium samples were obtained from both lab-scale and intermediate-scale bioreactors. Preceding LC analysis, all cell culture samples were conditioned by centrifugation, to remove all cells, and filtration through a 0.22 μm, Millex-GV syringe-driven filter (Millipore, Bedford, Mass.). Additionally, prior to injection on-column, each DS standard was diluted with 0.05% TFA in water to 0.20 μg/μL via a two-step dilution and each conditioned sample was diluted with 0.05% TFA in water by a factor of 10×. All samples diluted for LC/MS were then maintained at 4° C. in the sample manager.

Reversed-Phase Ultra-High Performance Liquid Chromatography (RP-U-HPLC). An ACQUITY ultraperformance liquid chromatography (UPLC®) system (Waters Corp., Milford, Mass.) was used for all chromatographic analyses. The ACQUITY system was equipped with a column heater, which has an upper temperature limit of 65° C.; a tunable, dual-wavelength ultraviolet/visible (UV/Vis) detector; and a sample manager with sample cooling capabilities. The ACQUITY was equipped with the system's original solvent mixer, which has a volume of 50 μL. The sample manager was maintained at 4° C. throughout all experiments. For all experiments described within, a Zorbax Poroshell 300SB-C3 microbore column (1.0×75 mm, 5 μm particles, Agilent Technologies, Inc. Palo Alto, Calif.) was used unless indicated otherwise. The chromatographic method used during both method optimization and in the final method employed a linear gradient using 0.05% (v/v) TFA in water (mobile phase A) and 0.05% TFA (v/v) in acetonitrile (mobile phase B). During method development, isopropanol was evaluated as an additive for mobile phase B at varying percentages, as a modifier to improve the chromatographic separation. However, isopropanol did not exhibit any effects on the chromatography that improved either the resolution or MS signal and, therefore, was not pursued during method optimization. In all experiments, the UV absorbance was monitored at 214 and 280 nm. In order to keep the back pressure at approximately 1000 psi or greater, which was necessary for the optimal operation of the Acquity pumps, 0.0025″ ID× 1/16″ OD PEEK™ Polymer Tubing (Upchurch Scientific, Inc., Oak Harbor, Wash.) was used for all external tubing requirements, including after the UV detector and as an inlet into the mass spectrometer. Gradients with varying steepness, several flow rates, and different column temperatures were tested during method optimization.

Mass Spectrometry. For all on-line LC/MS analyses, a high-resolution Q-Tof-2 mass spectrometer (Micromass Technologies, Waters Corp., Milford, Mass.) was used. The Q-Tof-2 has a hybrid quadrupole time-of-flight (QTOF) mass analyzer equipped with a Z-spray ion source for electrospray ionization. For all experiments, the collision energy of the Q-Tof-2 was set to 10 eV; the desolvation and ion source block temperatures were set to 275° C. and 115° C., respectively; the ESI capillary voltage was set to 3000 V; and the pusher cycle time was set to 88 μseconds. For all analyses, ions were detected over a m/z range of 500-4300 and a total scan time of 2 seconds (accumulated signal for 1.9 s and had an inter-scan delay to 0.1 s) was utilized. The following MS profile was used: dwell time at m/z 667 of 20%, a ramp time from m/z 667 to 2130 of 60%, and dwell time at m/z 2130 of 20%, where the indicated percentage was the percent of total scan time. For each MS acquisition, an MS method was created to vary the cone voltage based on the elution time of the sample components over the total MS acquisition time of 27 minutes. During and post elution of the intact mAb, which varied for each mAb and was reflected in the tailored MS method, the cone voltage was set to 60 V for optimum intensity and signal-to-noise. During elution of all components preceding the elution of the intact mAb, the cone voltage was set to 37 V. Prior to all on-line experiments, the Q-Tof-2 instrument was cleaned, prepared, mass calibrated with NaI, and maintained using a protocol developed at Wyeth BioPharma Development (Rouse, et al., 2005).

For all on-line experiments, an external, 6-port switching valve (EV700-100-WA, Valco Instruments Company Inc., Houston, Tex.) was used to switch the flow being introduced into the Q-Tof-2 between the LC effluent and a performance assessment solution, which was used to verify instrument mass accuracy and resolution. In the optimized method, the effluent flow rate was 100 μL/minute, so flow splitting was not required prior to MS analysis. In the optimized LC/MS method, the inflows into the Q-Tof-2 were performance assessment solution from 0 to 4 minutes, LC effluent from 4 to 22 minutes, and performance assessment solution from 22 to 27 minutes. The performance assessment solution comprised bovine insulin at 1 μmol/L and bovine trypsinogen at 2 μmol/mL in 50:50 acetonitrile:water (v/v) with 2% (v/v) formic acid. The performance assessment solution was flowed at 10 μL/minute and delivered using a Pump 22 syringe pump (Harvard Apparatus, South Natick, Mass.).

Software. All chromatographic data were acquired and processed using Waters Empower Pro software (version 5.00, Waters Corp., Milford, Mass.). MassLynx 3.5 for NT (Micromass MS Technologies, Waters Corp., Milford, Mass.) was the Q-Tof-2 software for instrument control, data acquisition, calibration, and mass spectrum processing. Probabilistic maximum entropy analysis (MaxEnt-1 module, Micromass MS Technologies, Waters Corp., Milford, Mass.) was used to deconvolute the multiply-charged mass data (that are typical for an ESI mass spectrum) into a zero-charge mass spectrum. This software enabled straightforward elucidation of molecular mass values for the different protein isoforms without prior knowledge of the protein species present in the sample. Protein Analysis Worksheet (PAWS), version 2000.06.08 for Windows 95/98/NT/2000 (Genomic Solutions, Ann Arbor, Mich.), was used for the determination of theoretical mass values (average mass) for intact proteins and subunits based on the predicted amino acid sequences and PTMs.

### Example 2

### Optimization of Method for Detecting Polypeptides

RP-HPLC method for the direct analysis of mAbs included the exploration of gradients with varying steepness, several flow rates, and different column temperatures to achieve the best chromatographic separation and ensure high sensitivity and compatibility with ESI-QTOF MS. In addition, a variety of columns were evaluated in the development of the low-flow, RP-HPLC method. The ZORBAX Poroshell 300SB-C8 and 300SB-C3 columns and the BioSuite pPhenyl RP column were identified as suitable columns for the characterization of mAb therapeutic candidates and the analysis of complex samples containing mAbs. After initial evaluations, the Poroshell 300SB-C3 column was determined to be a superior choice over the Poroshell 300SB-C8 and the BioSuite pPhenyl RP columns because it provided better peak shape for a number of intact mAbs that were tested on these columns.

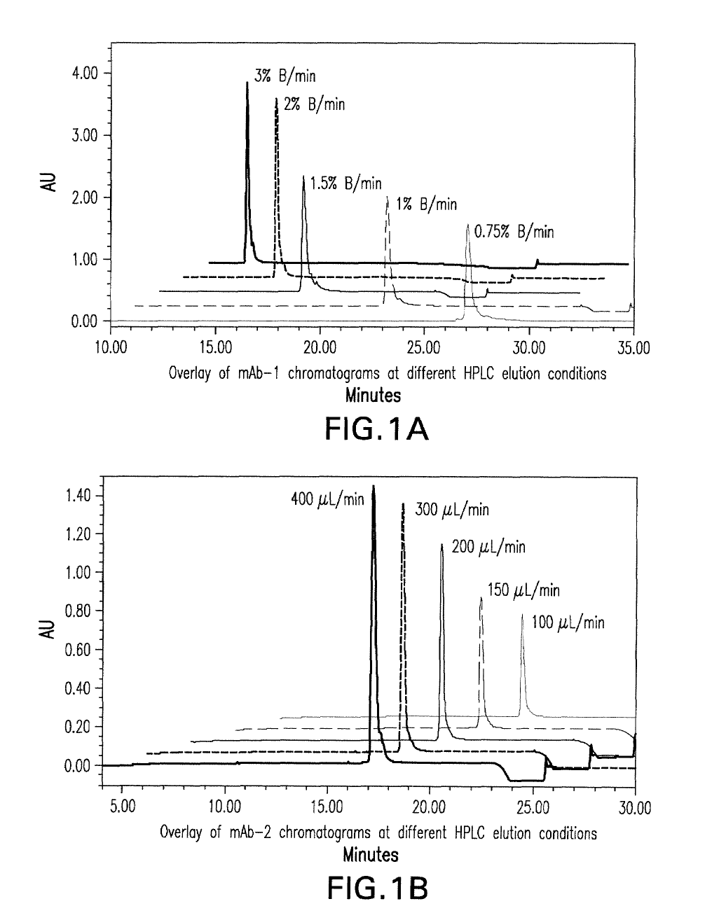

To determine the optimal linear gradient for the chromatographic elution of mAbs, experiments were performed on mAb-1, mAb-2, and mAb-3 with gradient slopes of 0.067% mobile phase B (B)/min to 5% B/min. An ideal and inclusive range of the analytical linear gradient for the analysis of mAbs and process-related species was determined to be 17% B to 42% B. In FIG. 1A, five representative chromatograms for mAb-1, with gradient slopes of 3% B/min, 2% B/min, 1.5% B/min, 1% B/min, and 0.75% B/min over the analytical range, are highlighted. In each of the separations, the flow rate was fixed at 100 μL/min. The chromatograms in FIG. 1A showed that as the gradient slope was increased, the peaks for mAb-1 increase in peak height and the elution time of mAb-1 was decreased, which enabled shorter chromatographic separation. However, this compression in peak shape lead to a loss of chromatographic resolution and an inability of the method to either resolve the characteristic “back shoulder” of the mAbs from its main peak or resolve close-eluting, product-related species from the mAbs. Therefore, a gradient of 1.5% B/min was determined to be an optimum balance for fast HPLC performance and resolution of product related species.

To determine the optimal flow rate for the chromatographic elution of mAbs, experiments were performed on mAb-1, mAb-2, and mAb-3 with flow rates from 100 μL/min to 500 μL/min. Because of the properties of the Poroshell particles, this microbore 300SB-C3 column, with an internal diameter of 1.0 mm, was expected to perform well at faster-than-usual flow rates. Typically, microbore columns operate optimally at flow rates of 40 to 60 μL/min. In FIG. 1B, five representative chromatograms for mAb-2, at 100 μL/min, 150 μL/min, 200 μL/min, 300 μL/min, and 400 L/min, are highlighted. In each of the separations, the gradient slope was fixed at 1.5% B/min. As depicted in FIG. 1B, the peak heights for the representative mAb were significantly increased as the flow rate was decreased and as the gradient slope remained constant. In addition, there was a loss of overall peak area, MS response, and MS signal-to-noise for the mAb peak as the flow rate was increased. Therefore, from the chromatographic profiles and MS quality, 100 μL/min was determined to be the optimal flow rate for an on-line RP-HPLC/ESI-QTOF MS method.

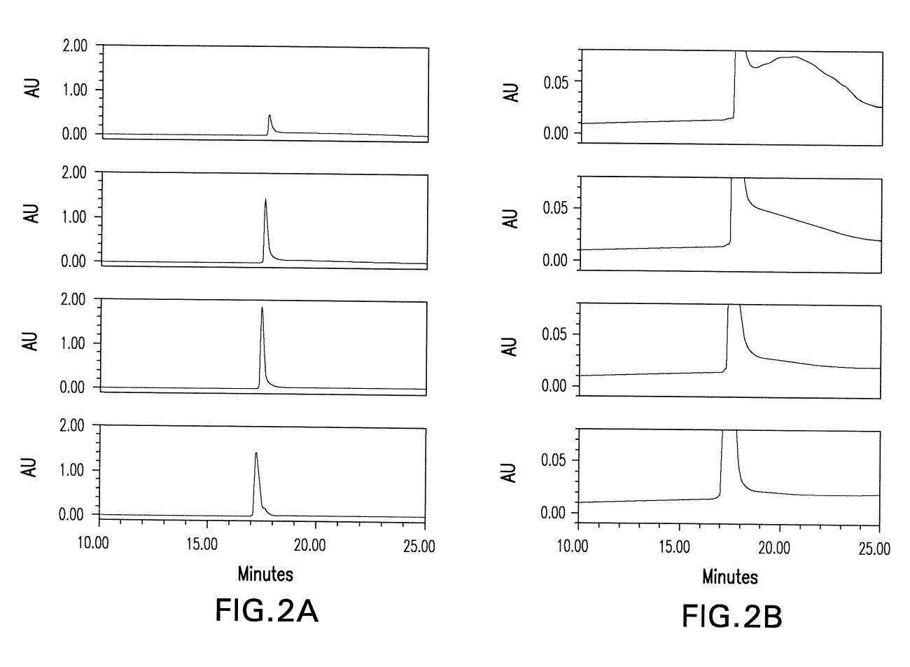

The third critical variable that was investigated for optimal mAb elution was the column temperature employed during separation. To determine the optimal column temperature for the chromatographic elution of mAbs, experiments were performed on mAb-1, mAb-2, and mAb-3 with column temperatures from 35° C. to 65° C. In FIG. 2, four representative chromatograms for mAb-2, at 35° C., 45° C., 55° C., and 65° C. are highlighted at full peak height (FIG. 2A) and at a zoom-in of the elution baseline (FIG. 2B). From the chromatograms exhibited in FIG. 2A, it was apparent that the peak height was increasing as the temperature was increased from 35° C. to 55° C. and then decreased slightly as the column temperature was increased to 65° C. However, as presented in Table 1, the peak area for mAb-1 continued to increase as the column temperature was raised to 65° C. This continued increase was indicative of the efficient analyte and particle interaction that occurs as the column temperature was raised. To support this reasoning, the zoomed-in chromatograms in FIG. 2B depict the undesirable peak shape of mAb-2 when the column temperature was varied from 35° C. to 55° C. Under these conditions, the detected chromatographic peak signal did not return to baseline. However, when a column temperature of 65° C. was used, the eluting chromatographic signal efficiently returned to baseline. Therefore 65° C. was determined to be the optimal column temperature for an efficient, baseline separation of mAbs.

TABLE 1
Chromatographic peaks at 214 nm
for RP-HPLC/UV analysis of mAb-1

| Temperature (° C.) | Peak Area (214 nm, AU) |
|---|---|
| 35 | 7.51 × 10⁶ |
| 45 | 2.04 × 10⁷ |
| 55 | 2.28 × 10⁷ |
| 65 | 2.38 × 10⁷ |

Taking the described experiments into consideration, the optimal low-flow RP-HPLC method, employing the ZORBAX Poroshell 300SB-C3 column and having compatibility with ESI-QTOF MS, was determined to employ a gradient with a rise of 1.5% mobile phase B/min (in the analytical segment), a flow rate of 100 μL/min, and a column temperature of 65° C. To allow for the initial retention of more hydrophilic species, the initial injection loading conditions were at 100% mobile phase A and to enable a more high-throughput method, a step gradient was used to rapidly go from the initial conditions of 0% B to the start of the analytical gradient at 17% B. The overall time from injection to injection was optimized to 30 min. In the optimized method flow-splitting was not required prior to MS analysis, as the flow rate was optimal for the ESI-QTOF MS conditions typically used in the lab. The gradient for the optimized chromatographic conditions used in this work was are indicated in Table 2, which shows the chromatographic peaks at 214 nm for RP-HPLC/UV analysis of mAb-1 at 35, 45, 55, and 65° C.

TABLE 2
Chromatographic Conditions Used

| Time | Flow Rate | Mobile | Mobile |
|---|---|---|---|
| (minutes) | (μL/minute) | Phase A (%) | Phase B (%) |
| Initial | 100 | 100.0 | 0.0 |
| 3.00 | 100 | 100.0 | 0.0 |
| 5.00 | 100 | 83.0 | 17.0 |
| 21.67 | 100 | 58.0 | 42.0 |
| 23.00 | 100 | 0.0 | 100.0 |
| 23.10 | 300 | 0.0 | 100.0 |
| 25.00 | 300 | 0.0 | 100.0 |
| 25.10 | 300 | 100.0 | 0.0 |
| 29.00 | 300 | 100.0 | 0.0 |
| 29.10 | 100 | 100.0 | 0.0 |
| 30.00 | 100 | 100.0 | 0.0 |

### Example 3

Characterization of drug substance (DS).

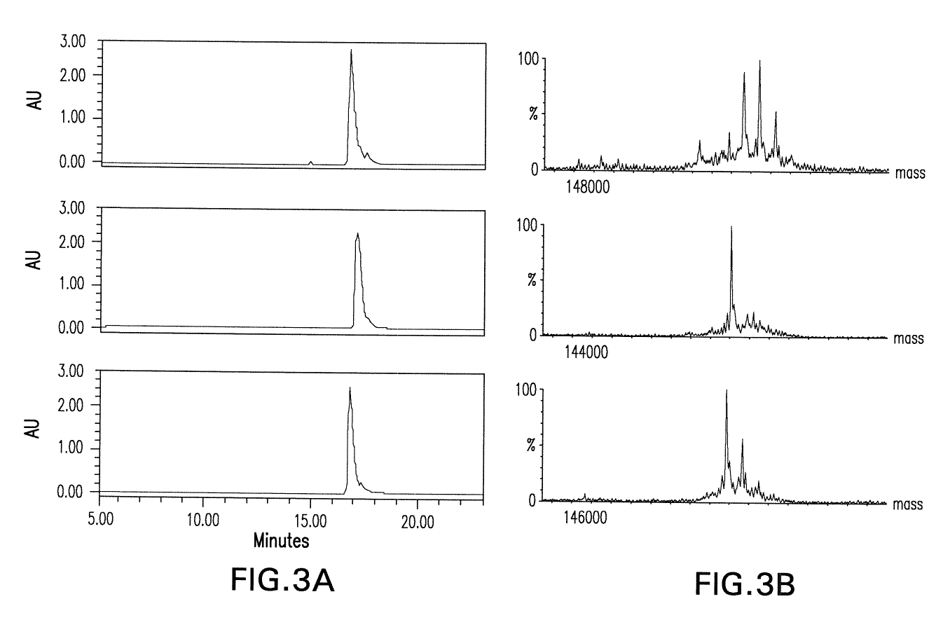

To demonstrate the utility and performance of the Poroshell 300SB-C3 RP-HPLC/ESI-QTOF MS method, the DS for three mAbs (mAb-1, mAb-2, and mAb-3) were analyzed. The UV chromatogram for each mAb is presented in FIG. 3A. For each DS, 5 μg (or 34 μmol) of mAb was injected on to the column. The chromatograms showed that there was one major protein component in each of the three DS and this component was the expected intact mAb. The chromatographic peak for each of the mAbs had a good shape and there was only minimal peak tailing. Also, each mAb eluted at a retention time and mobile phase composition that was similar to that of the other mAbs. Incidentally, the characteristic “back shoulder” that was present, yet slightly different, for each mAb had the same MS profile as the main peak. Additionally, this back shoulder did not appear to be due to a thermal degradation of the mAb at the high column temperature. The characteristic “back shoulder” did not substantially change when the column temperature was varied from 60° C. to 90° C., as determined by experiments performed on a prototype column heater for the Acquity. The slight separation that leads to the distinct back shoulder potentially may result from slightly different conformations of the mAb, which may be partially resolved by the method.

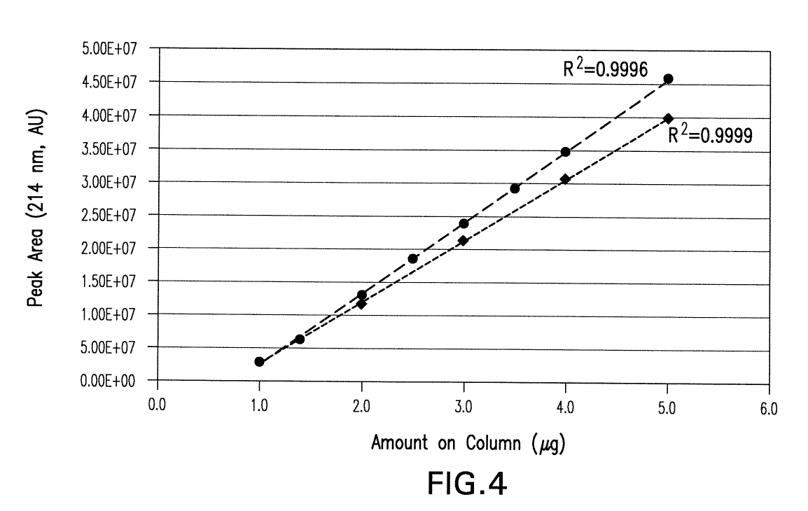

The corresponding deconvoluted, zero-charge mass spectrum for the detected chromatographic peak of each mAb DS is presented in FIG. 4B and the mass assignments of the mAb-1, mAb-2, and mAb-3 isoform species observed by Poroshell 300SB-C3 RP-HPLC/UV/ESI-QTOF MS analyses are presented in Table 3.

TABLE 3
Mass Assignments of the Observed Species

| | | Theoretical | Observed | Mass Error | |
|---|---|---|---|---|---|
| Sample | Isoform^{a} | Mass (Da)^{b} | Mass (Da)^{c} | (ppm) | Abundance^{d} |
| mAb-1 | G0F/G0F | 149,522.8 | 149,526.4 | 24 | Major |
| | G0F/G1F | 149,684.9 | 149,687.3 | 16 | Major |
| | G1F/G1F | 149,847.1 | 149,849.3 | 15 | Major |
| | Man₅/Man₅ | 149,066.3 | 149,071.1 | 32 | Minor |
| | G0F/G0 | 149,376.6 | 149,378.9 | 15 | Minor |
| | Unoccupied/Man₅ | 147,849.2 | 147,849.4 | 1 | Trace |
| | Unoccupied/G0F | 148,077.4 | 148,074.3 | 21 | Trace |
| | Unoccupied/G1F | 148,239.6 | 148,246.5 | 46 | Trace |
| mAb-2 | G0F/G0F | 145,414.1 | 145,414.2 | 1 | Major |
| | G0F/G1F | 145,576.3 | 145,579.1 | 19 | Minor |
| | G0F/G0F+HisSer^{e} | 145,638.4 | 145,640.5 | 14 | Minor |
| | G0F/G0F+AlaHisSer^{e} | 145,709.5 | 145,707.0 | 17 | Minor |
| mAb-3 | G0F/G0F | 147,370.4 | 147,372.0 | 11 | Major |
| | G0F/G1F | 147,532.5 | 147,534.4 | 13 | Minor |
| | G1F/G1F | 147,693.3 | 147,699.8 | 44 | Minor |
| | Man₅/Man₅ | 146,914.0 | 146,916.8 | 19 | Trace |
| | G0F/Man₅ | 147,142.2 | 147,136.3 | 40 | Trace |
| | G0F/G0F−GlcNAc | 147,167.2 | 147,171.3 | 28 | Trace |
| | G0F/G0 | 147,224.4 | 147,228.0 | 24 | Trace |

^{a}Abbreviations for oligosaccharides observed on mAbs: G0F—core-fucoyslayted, asialo-biantennary complex-type structure with zero terminal galactose residues. G1F—core-fucoyslayted, asialo-biantennary complex-type structure with one terminal galactose residues. Man—mannose. Man₅—high mannose structure containing five man resides. G0—G0F structure with out the core-fucosylation. GlcNAc—N-acetylglucosamine. G0F-GlcNAc—G0F structure with only one terminal GlcNAc. Gal—galactose. Fuc—fucose.
^{b}Theoretical masses were calculated for intact mAb isoforms using PAWS for Windows 95/98/NT/2000 (2000.06.08) and are reported as average masses.
^{c}Observed masses were calculated using MaxEnt-1. Observed masses were calculated using MaxEnt-1. See Table D for details regarding MaxEnt-1 specifications. All observed mass values have a mass difference less than 50 ppm from the theoretical mass values, which is within specification for the analysis of large proteins on the Q-Tof-2.
^{d}Abundances are determined based on the detected distribution of mAb isoforms. Major = X ≧ 40% of base peak; Minor = 40% ≧ X ≧ 2%; Trace = Detectable and X < 2%.
^{e}NH₂-terminal amino acid residues of HisSer and AlaHisSer are observed for the mAb-2 light chain.

For each of the mass spectra presented in FIG. 3B, the major, minor, and trace isoforms were detectable and readily resolvable by the microbore RP-HPLC/ESI-QTOF MS method. In addition, each identified isoform had an observed mass that had a mass difference of less than 50 ppm from the calculated, theoretical mass for that isoform species. As outlined in Table 3, the major driver for the number of mAb isoforms was the micro-heterogeneity associated with the N-linked oligosaccharide present at the amino acid sequence consensus site of each heavy chain. This observed heterogeneity was typical for mAbs and the varying isoform distributions for each mAb was representative of the different cell culture processes and cell line phenotype. For mAb-2, there was additional heterogeneity that had been previously identified and described as alternative NH2-terminal amino acid extensions on the light chain, which result from alternate cleavage of the signal peptide sequence by the signal peptidase during protein translocation and were dependent on both the amino acid sequence of the signal peptide and the mature protein (McClellan, J. E., et al., 2004, 1^{st} Symposium on the Practical Applications of Mass Spectrometry in the Biotechnology and Pharmaceutical Industries. Boston Mass. September 17-18; and McClellan, J. E. et al., 2005, 9th Symposium on the Interface of Regulatory and Analytical Sciences for Biotechnology, Washington, D.C. January 9-13).

This microbore method had a linear response over a range from approximately 1 μg to 5 μg for most mAbs, which is depicted for mAb-1 and mAb-3 in FIG. 3. As a result of this linear response over this range, the method was quantitative and can be used to directly determine the concentration of mAbs in samples containing other proteins in addition to generating high quality mass spectral data on the mAbs. The slopes of the two calibration curves were slightly different for the two mAbs, as the absorptivity response was slightly higher for mAb-1 at 214 nm as compared to the response for mAb-3 because the molecule was slightly larger and had more covalent bonds along the peptide backbone, which absorb at 214 nm. Though the linear dynamic range of this method was limited to less than one order of magnitude for the amount of mAb on-column, the method had been useful for quantitative analysis of appropriately diluted DS; fractions from orthogonal chromatographic methods, such as weak cation exchange (wCEX)-HPLC and size exclusion (SE)-HPLC; and other complex mixtures. Furthermore, the method can quantitatively determine the amount of a mAb in a solution with concentration ranges of about 0.02 μg/L to about 1.0 μg/L when the system was equipped with a 50 μL sample loop or about 10.0 μg/L when the system was equipped with a 5 μL sample loop. Additionally, with the use of up to three sample injections for one chromatographic analysis, the method can reliably determine the amount of a mAb in a sample with a concentration as low as about 0.007 μg/L. While it requires prior knowledge of the sample's approximate concentration or multiple chromatographic analyses to assure the peak area was within the quantitative area, the method's effective, quantitative dynamic range was greater than three orders of magnitude, from about 0.007 μg/L to about 10.0 μg/L.

### Example 4

### Direct Analysis of Bioreactor Process Samples

Bioreactor process samples for mAb-1, mAb-2, and mAb-3, which were obtained from an intermediate-scale bioreactor process, were analyzed. The same optimized microbore LC/MS method used for the analysis of the DS in Section 3.2 was used for all three mAbs. Each bioreactor process samples was prepared as described in the Experimental section, with minimal sample handling and no expected sample speciation.

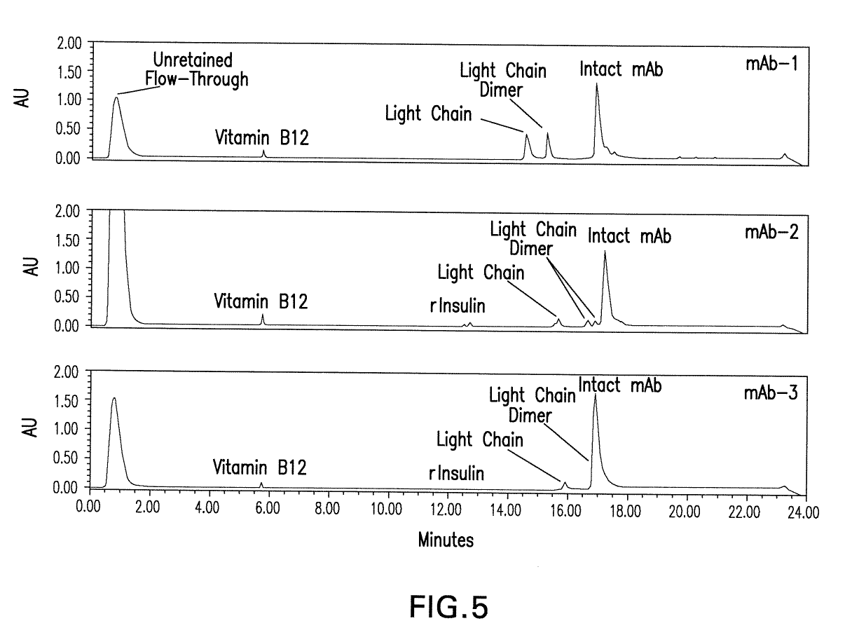

The direct analyses of each of these conditioned bioreactor process samples are presented in FIG. 5. As the labeled, RP-HPLC/UV chromatograms indicated, the method was capable of chromatographically retaining, separating, detecting, and identifying the intact mAb; product-related species, including free light chain and light chain dimer; and specified process-related biomolecules for each mAb sample. The two biomolecules that were detected by this method, vitamin B12 and recombinant insulin, were constituents added to the cell culture medium as part of the manufacturing process for each mAb. Interestingly, though the bioreactor scale and cell culture processes were similar, each bioreactor was sampled at similar time point, and centrifuged, filtered, and injected in the same manner, the chromatographic profile for each mAb was unique, with respect to peak area associated with the intact mAb and the peak area of the product related species. Additionally, there was a varied UV response in the magnitude of the chromatographically unretained components, the detected amount of vitamin B12, and the detected amount of recombinant insulin, which was not detected for the mAb-1 process when only 1.5 μL of the conditioned sample was injected.

A detailed list of the intact mAb and product-related species bioreactor components identified in FIG. 5, with their respective theoretical and observed masses, are presented in Table 4.

TABLE 4
Intact mAb and Components

| | | | Theoretical | Observed | Mass Error |
|---|---|---|---|---|---|
| Sample | Species | Isoform^{a} | Mass (Da)^{b} | Mass (Da)^{c} | (ppm) |
| mAb-1 | Free Light Chain | Cysteinylated | 24,088.8 | 24,088.7 | 4 |
| | | Glutathionlyated | 24,274.9 | 24,274.9 | 0 |
| | Light Chain Dimer | No Modifications | 47,937.2 | 47,936.7 | 10 |
| | Intact mAb | G0F/G0F | 149,522.8 | 149,523.0 | 1 |
| | | G0F/G1F | 149,684.9 | 149,685.8 | 6 |
| | | G1F/G1F | 149,847.1 | 149,846.2 | 6 |
| mAb-2 | Free Light Chain | Cysteinylated | 22,550.9 | 22,550.9 | 0 |
| | | Cysteinylated+Hex | 22,713.0 | 22,712.2 | 35 |
| | | Cysteinylated+HS^{d} | 22,775.1 | 22,774.1 | 44 |
| | | Cysteinylated+AHS^{d} | 22,846.2 | 22,845.5 | 31 |
| | Free Light Chain | No Modifications | 44,861.5 | 44,861.5 | 0 |
| | Intact mAb | +HS^{d} | 45,085.7 | 45,086.0 | 7 |
| | | +AHS^{d} | 45,156.8 | 45,155.7 | 24 |
| | | G0F/G0F | 145,414.1 | 145,414.5 | 3 |
| | | G0F/G1F | 145,576.3 | 145,574.0 | 16 |
| | | G0F/G0F+HS | 145,638.4 | 145,641.9 | 24 |
| | | G0F/G0F+AHS | 145,709.5 | 145,706.4 | 21 |
| mAb-3 | Free Light Chain | Cysteinylated | 22,812.1 | 22,811.8 | 13 |
| | | Glutathionlyated | 22,988.3 | 22,997.7 | 26 |
| | Light Chain Dimer | No Modifications | 45,384.0 | 45,383.6 | 9 |
| | Intact mAb | G0F/G0F | 147,370.4 | 147,372.8 | 16 |
| | | G0F/G1F | 147,532.5 | 147,535.8 | 22 |

^{a}Abbreviations for oligosaccharides observed on mAbs: G0F—core-fucoyslayted, asialo-biantennary complex-type structure with zero terminal galactose residues. G1F—core-fucoyslayted, asialo-biantennary complex-type structure with one terminal galactose residues. Gal or G—galactose. Fuc or F—fucose. Hex—hexose, generic nomenclature for galactose or mannose.
^{b}Theoretical masses were calculated for intact mAb isoforms using PAWS for Windows 95/98/NT/2000 (2000.06.08) and are reported as average masses.
^{c}Observed masses were calculated using MaxEnt-1. The following parameters were used for all MaxEnt-1 calculations: 1.000 Da/Channel (Resolution) and 67% (Minimum intensity ratio). For all mAbs, the Uniform Gaussian Width was set at 0.900, 0.850, and 0.750 Da for free light chain, light chain dimer, and intact mAb, respectively. All observed mass values are within mass accuracy specification for the analysis of large proteins on the Q-Tof-2.

In comparing the detected mAb isoforms observed in both the DS and conditioned media, the isoform distribution for both the DS and condition media were highly similar. This observation confirmed that this LC/MS method is capable of obtaining high quality mass spectral data from mixtures even as complicated as a conditioned media, which contains a large amount of salts and other small molecules that could adduct to the protein and interfere with the ESI-QTOF MS response. Also, it should be noted for the bioreactor samples from all three mAbs, the free light chain was present in both a cysteinylated and glutathionylated form and the light chain dimer was present in a fully oxidized form with all of the cysteines accounted for in disulfide bonds. Light chain with a free, reduced sulfhydryl was not observed for any of the bioreactor process samples.

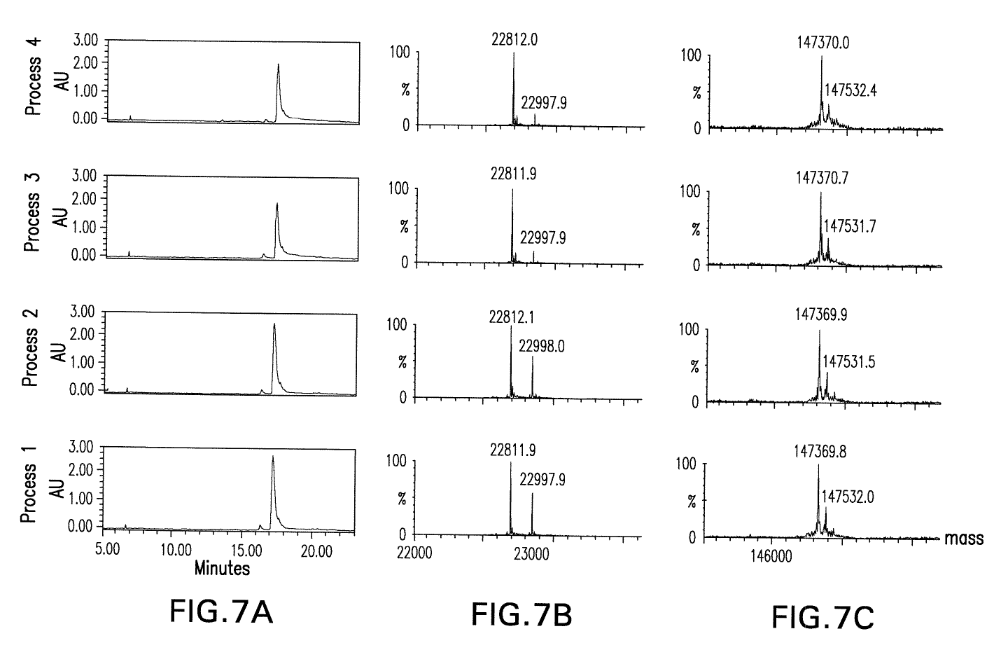

From the peak heights and peak area for each mAb presented in FIG. 7, it was possible to determine the relative productivity of each bioreactor process and the amount of mAb that had accumulated in the cell culture medium at the sampled time point. From the chromatographic profiles, and taking into account the molar absorptivity values for each mAb, the mAb-1 and mAb-3 processes have similar productivity, with the mAb-3 process being slightly more productive. Both processes were more productive than the process mAb-2. This determination was corroborated by complimentary data obtained by a Protein A-HPLC/UV affinity capture and elution method that is commonly used to determine the concentration of mAbs in cell culture medium. However, this Protein A method cannot contribute any information about the product quality or isoform distribution of the detected mAb. In comparing the detected mAb isoforms observed in both the DS and the conditioned media, the isoform distribution for both the DS and the conditioned media were highly similar. This observation confirmed that this LC/MS method is capable of obtaining high quality mass spectral data from mixtures even as complicated as a conditioned media, which contains a large amount of salts and other organic molecules that could adduct to the protein and interfere with the ESI-QTOF MS response.

As depicted in FIG. 5 and Table 3, the free light chain and light chain dimer were detected for each mAb in the cell culture medium, but no free heavy chain or heavy chain dimer was detected. This was consistent with the understanding of cellular expression, which states that light chains are necessary to transport mAb-related species across extra-cellular membranes (Dinnis and James, 2005, Biotech. Bioeng. 91(2):180-189). Therefore, species that are only comprised of heavy chains will not be excreted into the cell culture medium. Based on the chromatographic profiles, each bioreactor process generates a different amount of free light chain and light chain dimer in both absolute magnitude and relative to the amount of intact mAb that was generated. These observations may indicate that this method is capable of differentiating cell lines, cell line phenotypes, and bioreactor processes based on the chromatographic profile of the conditioned media. As such, this method has direct applicability for cell culture process optimization during the development of the biopharmaceutical manufacturing process.

### Example 5

### Application for Bioreactor Process Monitoring

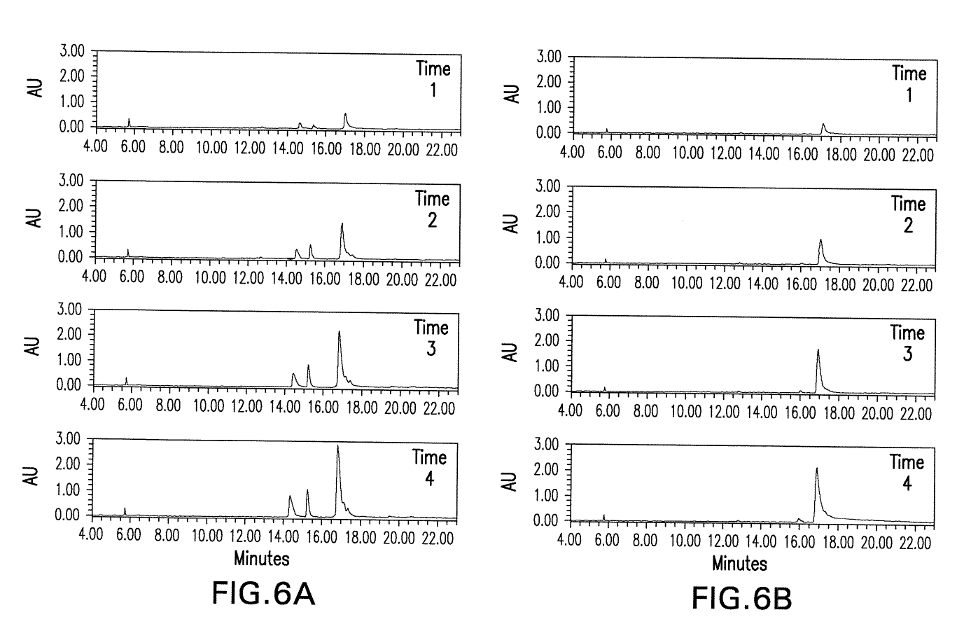

One useful application of this method for biopharmaceutical development is to monitor the changes in output of a bioreactor process over time. In FIG. 6, chromatographic profiles for mAb-1 (FIG. 6A) and mAb-3 (FIG. 6B) at the same four time points during the cell culture manufacturing process are presented. For mAb-1 and mAb-2, the accumulation of the intact mAb, free light chain, and light chain dimer occurred in a continual fashion throughout the process time points. It should be noted that for each mAb, there was a slight shift in retention time as the amount of mAb on column increased with later time points. While this shift was noteworthy, it did not impact the quantitative power of this assay and a similar trend was observed for all mAbs during method optimization, including for other mAbs that were not included in this manuscript.

In both examples, the peak shape of the three product-related components and the relative proportions of these components remained consistent throughout the entire bioreactor process, indicating that the bioreactor process was steadily produced and excreted the same products throughout all of the time points analyzed. Additionally, for both mAbs, a slight shift in the N-linked glycosylation profile was observed from the earliest sampled time point to the later bioreactor process time points. For both mAbs, a higher proportion of intact mAbs were observed to contain more galactosylated and sialylated N-linked oligosaccharides present at the amino acid sequence consensus site at the earlier time point than the intact mAbs observed at later time points.

### Example 6

### Application of Bioreactor Process Selection and Optimization

Another useful application of the micro-bore LC/MS method during cell culture process development is for the selection of the best manufacturing process. To illustrate this application, bioreactor process samples containing mAb-3, manufactured by four different bioreactor processes at small-scale, were analyzed and are presented in FIG. 7. The RP-HPLC/UV chromatographic profiles for the four processes (FIG. 7A) showed that each bioreactor process produced the same product species, even though there were differences in the amount of intact mAb produced. The deconvoluted, zero-charge mass spectra of the two main chromatographic peaks for free light chain and intact mAb are presented in FIG. 7B and FIG. 7C, respectively. The respective theoretical masses for the major isoforms of both components are depicted in FIG. 7.

The mass spectra from the four processes for mAb-3 showed that all of the same intact mAb isoforms were present and there was only a slight redistribution of the isoform's relative intensity among the four processes. Conversely, while the same two light chain species were present in each of the analyzed bioreactor process samples, there was a difference in the ratio of the cysteinlyated and glutathionylated species. In general, the four processes have highly similar outputs and produced mAbs with good quality that were all comparable to each other. Process 4 was determined to be the most productive, as it produced the largest amount of mAb in the same manufacturing time as the other processes. The information presented in this section had utility in defining the best manufacturing process for mAb-3 during early clinical development.

### Example 7

### Direct Analysis of Vitamins in Culture Media

Cell culture media samples were obtained from lab-scale, intermediate-scale, and full-scale bioreactors. Samples were injected on column undiluted and were maintained at 8° C. in the sample manager of the ACQUITY system.

An ACQUITY® ultra performance liquid chromatography (UPLC®) system (WATERS Corp., Milford, Mass.) was used for all Chromatographic analyses. The ACQUITY® system was equipped with a column heater, which had an upper temperature limit of 65° C., a Photo-Diode Array ultraviolet/visible (UV/Vis) detector, and a sample manager with sample heating/cooling capabilities. The ACQUITY® was equipped with the system's original solvent mixer, which had a volume of 50 μL. The sample manager was maintained at 8° C. throughout all experiments. For all experiments described within, a HSS T3 column (2.1×150 mm, 1.8 μm particles, Waters Corp., Milford, Mass.) was used. The column was maintained at 45° C. throughout the analysis. The chromatographic method employed a mix mode gradient using 20 mM ammonium acetate, pH 5.8, in water (mobile phase A) and 60/40 acetronitrile/15 mM ammonium acetate, pH 5.8, in water (v/v) (mobile phase B). The flow rate was held at 300 μL/min. The UV absorbance was monitored at 205, 262, 325, 345 and 445 nm. The different UV wavelengths were used to maximize the sensitivity of individual vitamins. For use with mass spectrometry, the ammonium acetate concentration was lowered to 5 mM in mobile phases A and B.

Due to an interfering peak in the conditioned medium at 205 nm, calcium pantothenate and biotin was quantified in uninoculated culture media. The gradient table for the optimized chromatographic conditions used in this work are indicated in Table 5:

TABLE 5
Chromatographic Conditions Used

| Time | Flow Rate | Mobile | Mobile | |
|---|---|---|---|---|
| (minutes) | (μL/minute) | Phase A (%) | Phase B (%) | Curve |
| Initial | 300 | 99.9 | 0.1 | Initial |
| 4.00 | 300 | 99.9 | 0.1 | 6 |
| 15.00 | 300 | 94.0 | 6.0 | 8 |
| 25.00 | 300 | 45.0 | 55.0 | 7 |
| 26.00 | 300 | 0.1 | 99.9 | 6 |
| 28.00 | 300 | 0.1 | 99.9 | 6 |
| 28.5 | 300 | 99.9 | 0.1 | 6 |
| 35.00 | 300 | 99.9 | 0.1 | 6 |

Spike and recovery studies were performed on Day 2 and Day 10 conditioned medium samples. These studies were performed to demonstrate the applicability of the method. The percent recovery results are indicated in Table 6:

TABLE 6
Percent Recovery Results

| Vitamin | Day 2 % Recovery | Day 10 % Recovery |
|---|---|---|
| Pyridoxine | 99.6 | 97.0 |
| Nicotinamide | 99.3 | 99.3 |
| Thiamine | 98.4 | 95.2 |
| Folic Acid | 98.8 | 99.0 |
| Vitamin B12 | 103.1 | 103.6 |
| Riboflavin | 105.0 | 103.7 |

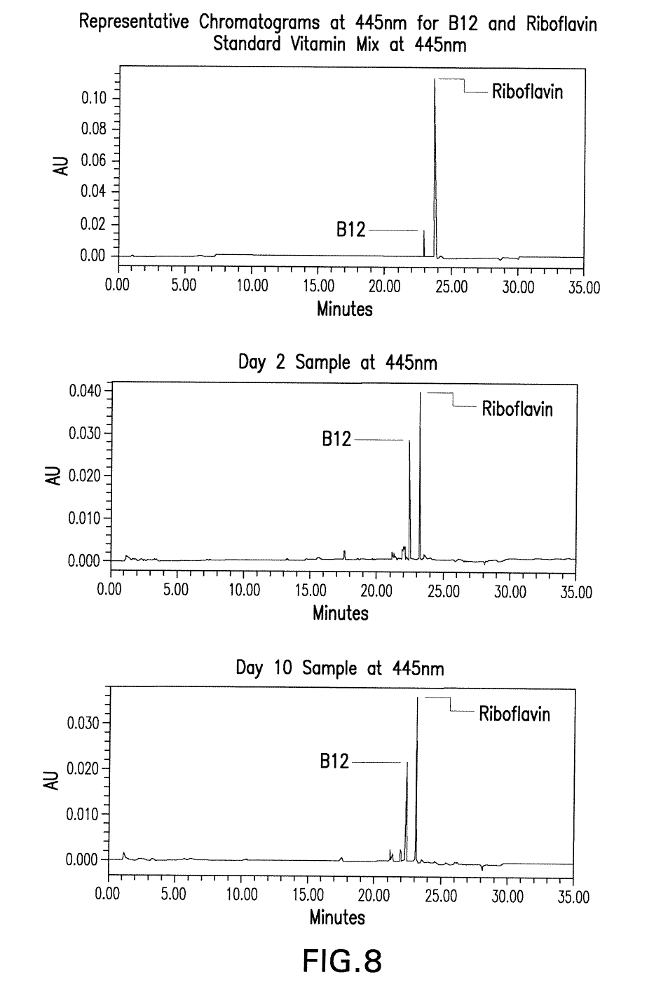

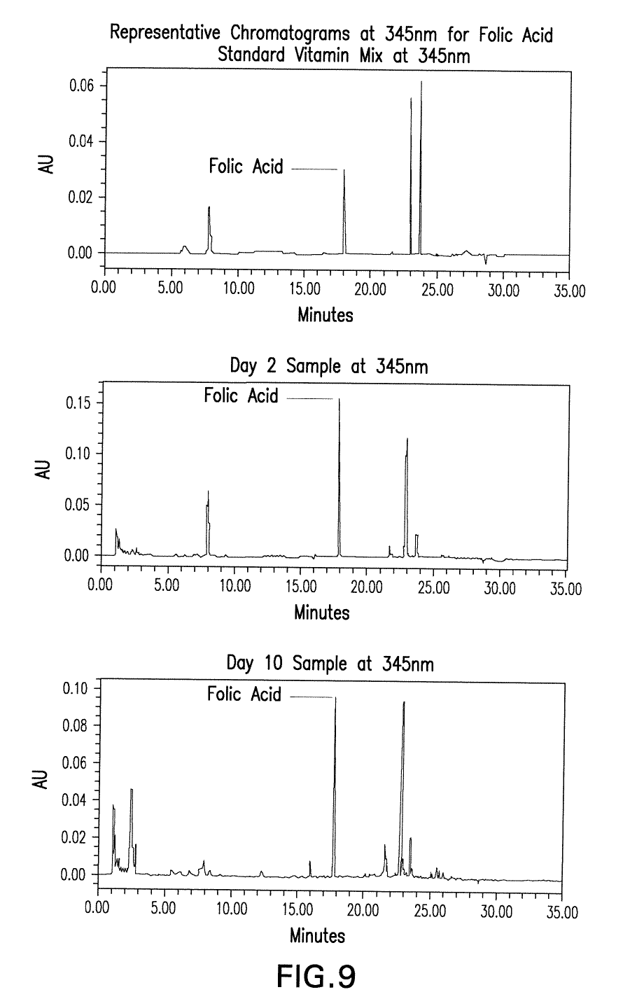

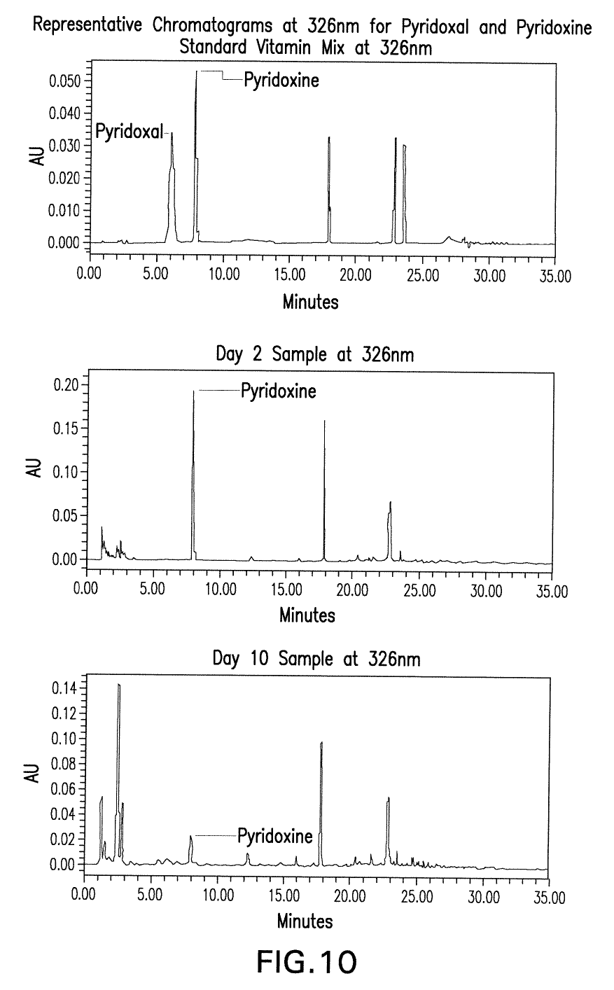

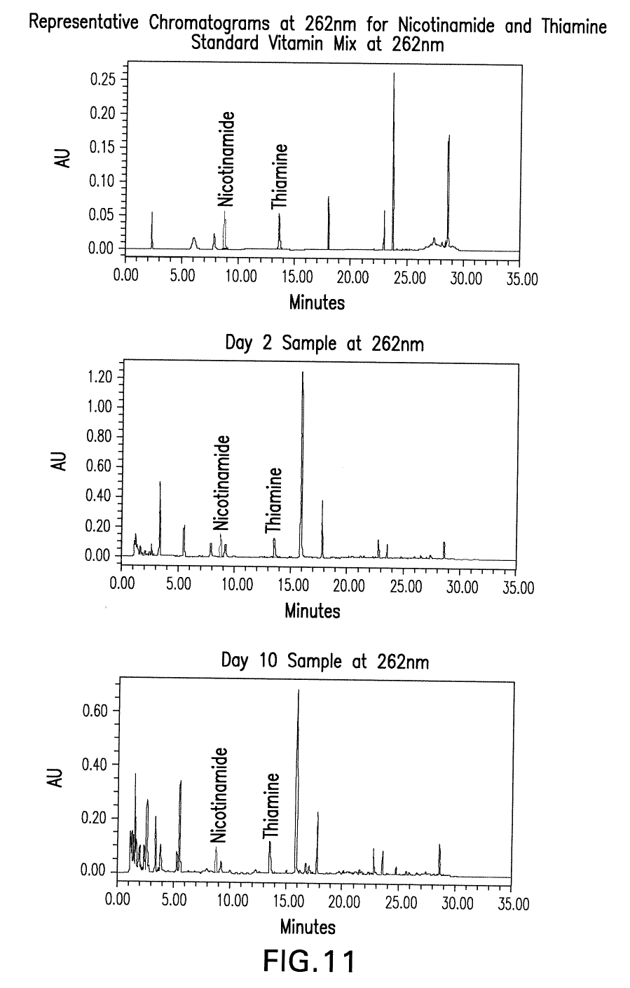

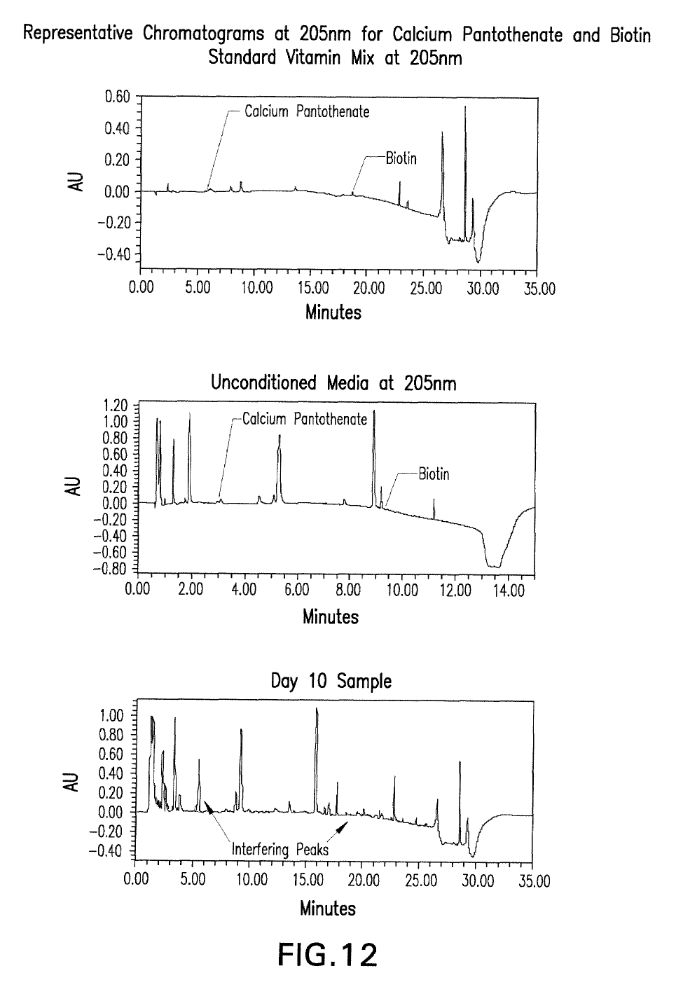

Representative chromatograms are shown in FIGS. 8, 9, 10, 11, and 12. All chromatographic data was acquired and processed using Waters Empower Pro software (version 5.00, Waters Corp., Milford, Mass.).

### Example 8

### Automated Mass Spectrometry of Ultra High Performance Liquid Chromatography Eluent for Analysis of Vitamin Content

The format of the RP-UPLC/PDA method for the direct analysis of vitamins in complex mixtures, such as cell culture medium, readily lends itself to on-line analysis by mass spectrometry. A number of mass spectrometers with electrospray ionization of atmospheric pressure chemical ionization can be used for analysis of the UPLC effluent, ranging from standard-resolution instruments such as quadrupole mass filter and quadrupole ion trap instruments or high-resolution quadrupole time-of-flight mass analyzers. All of these instruments have the appropriate sensitivity, selectivity, and scan rates to enable effective mass analysis of the eluting mixture components. In an on-line format, the RP-UPLC/PDA/MS method enables the detection and identification of both expected and unexpected species resolved by the chromatographic method.

As required for the UPLC/PDA/MS experiments, an external, 6-port switching valve (EV700-100-WA, Valco Instruments Company Inc., Houston, Tex.) can be used to switch the flow being introduced into the mass spectrometer between the liquid chromatography effluent and a performance assessment solution, which may be used to verify instrument mass accuracy and resolution. The method could be enhanced by automation of bioreactor process sampling, which can be readily accomplished by the use of either standard or customized robotics and automation solutions available to the biopharmaceutical industry. All chromatographic data can be acquired and processed using Waters Empower Pro software (version 5.00, Waters Corp., Milford, Mass.).

A number of embodiments of the invention have been described. Nevertheless, it will be understood that various modifications may be made without departing from the spirit and scope of the invention. Accordingly, other embodiments are within the scope of the following claims.

What is claimed:

1. A method of evaluating a process to produce a polypeptide preparation or evaluating an element of a process of producing a polypeptide preparation, the method comprising:
providing a cell culture sample or a process bioreactor sample comprising a polypeptide, wherein the cell culture sample or the process bioreactor sample has not been subjected to affinity chromatography, and
subjecting the cell culture sample or process bioreactor sample to a reverse-phase liquid chromatography, a size exclusion liquid chromatography, a normal-phase liquid chromatography, a hydrophobic interaction liquid chromatography, or an anion exchange liquid chromatography to determine the absence, presence, value or amount of a process characteristic in the sample,
thereby evaluating the process or element of the process of producing a polypeptide preparation.

2. A method of evaluating a process to produce a polypeptide preparation or evaluating an element of a process of producing a polypeptide preparation, the method comprising:
providing a cell culture sample or a process bioreactor sample comprising a polypeptide, wherein the cell culture sample or the process bioreactor sample has not been subjected to affinity chromatography,
subjecting the cell culture sample or process bioreactor sample to a liquid chromatography capable of detecting a process characteristic present in the cell culture sample or process bioreactor sample at a concentration of at least 0.007 μg/μL to determine the absence, presence, value or amount of a process characteristic in the sample,
thereby evaluating the process or element of the process of producing a polypeptide preparation.

3. The method of claim 1 or 2, further comprising providing a determination of whether the value determined for the process characteristic meets a preselected criteria.

4. The method of claim 1, wherein the element being evaluated is a culture process selected from pH, feeding condition, osmolarity, carbon dioxide level, shear force, agitation rate, temperature, oxidation, cell density, seeding density, timing, and sparge rate.

5. The method of claim 1, wherein the polypeptide preparation is a recombinant polypeptide preparation.

6. The method of claim 3, wherein the process characteristic is present in a preselected range.

7. The method of claim 1, wherein the method provides real time or near real time analysis of the sample.

8. A method of evaluating the absence, presence or amount of a polypeptide or a polypeptide isoform in a cell culture medium sample, the method comprising:
providing a cell culture medium sample, wherein the cell culture medium sample has not been subjected to chromatography,
subjecting the cell culture media sample to liquid chromatography (LC) to determine a process characteristic in the sample, wherein the concentration of the polypeptide or the polypeptide isoform in the cell culture medium is at least 0.007 mg/mL, to thereby evaluate the absence, presence or amount of the polypeptide or polypeptide isoform in the cell culture medium.

9. The method of claim 8, wherein the sample is obtained from a serum free cell culture medium.

10. The method of claim 8, wherein the cell culture medium sample has been previously conditioned.

11. The method of claim 8, wherein the liquid chromatography (LC) is selected from the group consisting of a reverse-phase LC, a size exclusion LC, a normal-phase LC, a hydrophobic interaction LC, an anion exchange LC, and a weak cation exchange LC.

12. The method of claim 8, wherein the method provides real time or near real time analysis of the sample.

13. A method of evaluating the absence, presence or amount of a vitamin in a cell culture medium sample, comprising:
providing a cell culture medium sample comprising a vitamin,
subjecting the cell culture media sample to an ultra high performance liquid chromatography (UHPLC), and
determining the absence, presence or amount of the vitamin in the sample.

14. The method of claim 13, wherein the cell culture medium sample has been previously conditioned.

15. The method of claim 13 or 14, wherein the UHPLC includes a sub-2 micrometer particle further comprising a C1 to C18 ligand capable of separating highly polar compounds.

16. The method of claim 15, wherein the sub-2 micrometer particle comprises a high strength silica core.

17. The method of claim 13, wherein the method provides real time or near real time analysis of the sample.

18. A method of evaluating a process to produce a polypeptide preparation, the method comprising:
providing a sample comprising a polypeptide, wherein the sample has not been subjected to affinity chromatography,
subjecting the sample to liquid chromatography, and
determining a value of a characteristic of the sample;
wherein the process to produce a polypeptide preparation is evaluated by determining whether the value determined for the characteristic of the sample meets a preselected criterion.

19. The method of claim 18, wherein the sample is from a process bioreactor.

20. The method of claim 18, wherein the liquid chromatography is selected from a reverse-phase liquid chromatography, a size exclusion liquid chromatography, a normal-phase liquid chromatography, a hydrophobic interaction liquid chromatography, an anion exchange liquid chromatography, and an ultra high performance liquid chromatography.

21. The method of claim 18, wherein the sample comprises a recombinant polypeptide.

22. The method of claim 18, wherein the value of the characteristic of the sample is a polypeptide isoform.

23. The method of claim 22, wherein the value of the polypeptide isoform is as low as about 0.007 μg/L.

24. The method of claim 18, wherein the method provides real time or near real time analysis of the sample.

25. A method of determining the absence, presence, or amount of a vitamin in a sample from a cell culture medium, the method comprising
subjecting the sample from the cell culture medium to an ultra performance liquid chromatography.

26. The method of claim 25, wherein the cell culture medium sample is conditioned.

27. The method of claim 25, wherein the ultra high performance liquid chromatography is performed with a sub-2 micrometer particle further comprising a C1 to C18 ligand capable of separating highly polar compounds.

28. The method of claim 27, wherein the sub-2 micrometer particle comprises a high strength silica core.

29. The method claim 25, wherein the method is a near real-time method.
